(19) United States
(12) Patent Application Publication
(10) Pub. No.: US 2023/0255467 A1
(43) Pub. Date: Aug. 17, 2023

(54) DIAGNOSTIC IMAGING DEVICE, DIAGNOSTIC IMAGING METHOD, DIAGNOSTIC IMAGING PROGRAM, AND LEARNED MODEL

(71) Applicants: JAPANESE FOUNDATION FOR CANCER RESEARCH, Tokyo (JP); AI Medical Service Inc., Tokyo (JP)

(72) Inventors: Yohei IKENOYAMA, Tokyo (JP); Sho SHIROMA, Tokyo (JP); Toshiyuki YOSHIO, Tokyo (JP); Tomohiro TADA, Tokyo (JP)

(21) Appl. No.: 17/997,028

(22) PCT Filed: Apr. 15, 2021
(86) PCT No.: PCT/JP2021/015555

§ 371 Date: Oct. 25, 2022

(30) Foreign Application Priority Data
Apr. 27, 2020 (JP) 2020-078601

(51) Int. Cl. A61B 1/273; G06T 7/00; G16H 50/20; A61B 1/00

(52) U.S. Cl. CPC A61B 1/273; G06T 7/0012; G16H 50/20; A61B 1/00045; G06T 2207/10016; G06T 2207/10068; G06T 2207/20081; G06T 2207/30096

(57) ABSTRACT

Provided are a diagnostic imaging device, a diagnostic imaging method, a diagnostic imaging program and a learned model, which can improve the diagnostic accuracy for esophagus cancer in esophagogastroduodenoscopy. This diagnostic imaging device comprises: an endoscopic image acquisition unit for acquiring an endoscopic moving image showing the esophagus of a person being tested; an estimation unit for estimating the location of esophagus cancer that is present in the acquired endoscopic moving image, using a convolutional neural network that has learned from esophagus cancer images as training data, the esophagus cancer images having been obtained from esophaguses affected by esophagus cancer; and a display control unit that superimposes on the endoscopic moving image an estimated location of the esophagus cancer and a certainty factor serving as an index for the probability that esophagus cancer is present at that location.

### TECHNICAL FIELD

The present invention relates to an image diagnosis apparatus, an image diagnosis method, an image diagnosis program and a learned model.

### BACKGROUND ART

Esophageal cancer is the eighth most common cancer worldwide among all cancers and has the sixth highest cancer-related mortality rate, killing more than 500,000 people annually. Esophageal squamous cell carcinoma is the most common form of esophageal cancer in South America and Asia (including Japan). Advanced esophageal cancer has a poor prognosis, but superficial esophageal cancer can be treated with minimally invasive procedures such as endoscopic resection if detected at an early stage and have a good prognosis. Therefore, early detection of superficial esophageal cancer is the most important issue.

The development of endoscopic techniques has increased the early detection of esophageal cancer, which has led to improved prognosis and minimally invasive treatment that preserves organs. Furthermore, with the development of endoscopic submucosal dissection (ESD), treatment of early esophageal cancer has become minimally invasive. However, according to Japanese guidelines for the diagnosis and treatment of esophageal cancer, the indication for ESD is limited to esophageal cancer that has invaded the mucosal layer, making it important to detect and diagnose esophageal cancer at an early stage.

However, it is difficult to detect superficial esophageal cancer by white light imaging (WLI), in which white light is irradiated onto the subject's esophagus for observation, even if endoscopy (EGD: Esophagogastroduodenoscopy) is performed. On the other hand, Narrow Band Imaging (NBI: Narrow Band Imaging) is useful for detecting superficial esophageal carcinoma, but it is difficult to detect it using only white light imaging (WLI), in which the subject's esophagus is irradiated with narrow-band light. However, the detection rate for inexperienced endoscopists is reported to be as low as 53%, even when using narrow-band light observation.

This is because esophageal cancer occurs as a flat lesion with little color variation and almost no irregularity, and these findings are difficult to recognize as a lesion without skill. In addition, because the background mucosa is often accompanied by inflammation, inexperienced endoscopists tend to confuse the inflamed mucosa with esophageal cancer, making the determination of cancerous lesions even more difficult. Thus, compared to colorectal cancer, which is characterized by polyps, esophageal cancer is still more difficult to diagnose endoscopically and more advanced diagnostic techniques are required in the field of endoscopic diagnosis, even though it is generally referred to as the digestive tract.

In addition to improvements in endoscopic instruments, biochemical methods are being developed as examination techniques. One such method is the highly sensitive detection of esophageal cancer using (Lugol's) iodine staining, in which iodine liquid is sprayed into the esophageal lumen. Specifically, in a test method in which the multiple iodine unstained area (the area that does not stain brown and shows yellowish-white when iodine liquid is sprayed into the esophageal lumen) is used as a biomarker, the incidence of esophageal cancer and head and neck cancer is reported to be higher in subjects (patients) with multiple iodine unstained areas in the esophagus after iodine staining, in comparison with subjects (patients) without multiple iodine unstained areas.

Since multiple iodine unstained areas are associated with heavy smoking, alcohol consumption, and low intake of green and yellow vegetables, and since multiple iodine unstained areas in the esophagus are said to result from the occurrence of TP53 mutations in the background epithelial cancer suppressor gene, and since, as mentioned above, subjects with multiple iodine unstained areas are at higher risk for esophageal and head and neck cancer, observation using iodine staining is suitable for precise screening for esophageal and head and neck cancer through endoscopic examination.

However, iodine staining has problems such as chest discomfort (side effects) and prolonged operation time, making its use in all cases impractical. It is desirable to use and select a very limited number of high-risk cases, such as those with a history of esophageal cancer or those with head and neck cancer complications. Further rapid and useful methods are needed for the early detection of esophageal cancer, such as high-precision testing methods that do not require iodine staining or testing methods that combine iodine staining when necessary.

Artificial Intelligence (AI) using deep learning has been developed in recent years and applied in the medical field. Furthermore, the development of convolutional neural networks (CNNs), which perform convolutional learning while maintaining the features of images input to AI, has dramatically improved the image diagnostic capability of computer-aided diagnosis (CAD) systems, which classify and judge learned images.

There have been various reports of deep learning-based image determination technology in the medical field, in which AI assists specialists in diagnosis, including radiological imaging, skin cancer classification, histological classification of pathological specimens, and colorectal lesion detection using ultra-magnifying endoscope. In particular, it has been proven that AI can achieve the same level of accuracy as specialists at the microscopic endoscopic level (see NPL 1). In dermatology, it has been also published that AI with deep learning capabilities can provide diagnostic imaging capabilities equivalent to those of specialists (see NPL 2), and patent literatures using various machine learning methods (see PTLS 1 and 2) also exist.

However, when still images are used as teacher data for training and the AI judges the still images taken during the examination, the AI cannot make a judgment unless still images are taken, so there are still some problems. Therefore, the AI cannot make a judgment unless a still image is taken, and it takes time to observe a large area with a still image. In addition, diagnostic imaging technology to detect high-risk cases of esophageal cancer by estimating the presence or absence of multiple iodine unstained areas, one of the biomarkers, has not yet been introduced in actual medical practice (actual clinical practice).

In summary, the requirements for future AI diagnostic assistive technologies are to provide real-time, precise image diagnosis assistance using moving images and to improve diagnostic accuracy by combining diagnosis with biomarker judgments related to cancer risk, in order to approach the comprehensive diagnostic technologies of endoscopic experts.

### CITATION LIST

### Patent Literature

PTL 1
Japanese Patent Application Laid-Open No. 2017-045341
PTL 2
Japanese Patent Application Laid-Open No. 2017-067489

### Non-Patent Literature

NPL 1
http://www.giejournal.org/article/S0016-5107(14)02171-3/fulltext, “Novel computer-aided diagnostic system for colorectal lesions by using endocytoscopy” Yuichi Mori et. al. Presented at Digestive Disease Week 2014, May 3-6, 2014, Chicago, Ill., USA
NPL 2
Nature, February 2017, Volume 1, Article, “Learning about skin lesions: enhancing the ability of artificial intelligence to detect skin cancer from images.” http://www.Natureasia.Com/ja-jp/nature/highlights/82762
NPL 3
Horie Y, Yoshio T, Aoyama K, et al. The diagnostic outcomes of esophageal cancer by artificial intelligence using convolutional neural networks. Gastrointest Endosc. 2018, 89: 25-32

### SUMMARY OF INVENTION

### Technical Problem

As mentioned above, it has been suggested that AI's diagnostic imaging capability in the medical field is as good as that of medical specialists in some cases. However, the technology to diagnose esophageal cancer in real time and with high accuracy using AI's diagnostic imaging capability has not yet been introduced into actual medical practice (actual clinical practice), and is expected to be put into practical use at an early date. In cancer imaging diagnosis, criteria based on the characteristics of cancer tissue, such as morphological features, tissue-derived biochemical biomarkers, and cell biological responses, are indispensable. Even with the diagnosis of gastrointestinal cancer by endoscopy, different organs require different AI diagnosis programs to design techniques and criteria optimized for each organ.

For example, flat esophageal cancer is a different form of cancer from colorectal cancer, which is easily detected by raised polyps, and is more difficult to detect, requiring new devices and techniques. Since there is a high possibility that the accuracy and judgment of the results obtained from medical equipment may change depending on the experience of the operator, the method to optimize the operating method of the endoscopist, who operates the equipment, as well as the functions related to image processing of the endoscope, should be considered among the devices and techniques. In other words, the extraction of unique features of each gastrointestinal cancer (esophageal cancer, gastric cancer, colorectal cancer, etc.) and the criteria for determining the pathological level of the cancer differ, and an AI program should be designed to suit the characteristics of each cancer type. In addition, functions to optimize operation when using the device and new technologies to evaluate mucosal characteristics such as biomarkers that express cancer risk as well as direct observation of the mucosa are also desired to be developed as useful combination technologies.

An object of the present invention is to provide an image diagnosis apparatus, an image diagnosis method and an image diagnosis program that can improve the diagnosis accuracy of esophageal cancer in an esophageal endoscope inspection.

### Solution to Problem

An image diagnosis apparatus according to the present invention includes: an endoscopic image acquisition section configured to acquire an endoscope video obtained by capturing an esophagus of a subject; an estimation section configured to estimate a position of an esophageal cancer present in the endoscope video acquired by using a convolutional neural network having been subjected to learning with an esophageal cancer image obtained by capturing an esophagus where an esophageal cancer is present as teacher data; and a display control section configured to display the position of the esophageal cancer estimated and a degree of certainty indicating a possibility of presence of the esophageal cancer at the position on the endoscope video in a superimposed manner.

An image diagnosis method according to the present invention includes: acquiring an endoscope video obtained by capturing an esophagus of a subject; estimating a position of an esophageal cancer present in the endoscope video acquired by using a convolutional neural network having been subjected to learning with an esophageal cancer image obtained by capturing an esophagus where an esophageal cancer is present as teacher data; and displaying the position of the esophageal cancer estimated and a degree of certainty indicating a possibility of presence of the esophageal cancer at the position on the endoscope video in a superimposed manner.

An image diagnosis program according to the present invention is configured to cause a computer to execute: an endoscopic image acquisition process of acquiring an endoscope video obtained by capturing an esophagus of a subject; an estimation process of estimating a position of an esophageal cancer present in the endoscope video acquired by using a convolutional neural network having been subjected to learning with an esophageal cancer image obtained by capturing an esophagus where an esophageal cancer is present as teacher data; and a display control process of displaying the position of the esophageal cancer estimated and a degree of certainty indicating a possibility of presence of the esophageal cancer at the position on the endoscope video in a superimposed manner.

A learned model according to the present invention includes is obtained through learning of a convolutional neural network with a multiple iodine unstained area esophagus image and a non-multiple iodine unstained area esophagus image as teacher data, the multiple iodine unstained area esophagus image being a non-iodine staining image obtained by capturing an esophagus where a multiple iodine unstained area is present without performing iodine staining, the non-multiple iodine unstained area esophagus image being a non-iodine staining image obtained by capturing an esophagus where no multiple iodine unstained area is present without performing iodine staining, the learned model being configured to cause a computer to estimate whether there is an association between an endoscopic image obtained by capturing an esophagus of a subject and an esophageal cancer, and output an estimation result.

### Advantageous Effects of Invention

According to the present invention, the diagnosis accuracy of esophageal cancer can be improved in esophageal endoscope inspection.

### BRIEF DESCRIPTION OF DRAWINGS

FIG. 1 is a block diagram illustrating a general configuration of an image diagnosis apparatus in a first embodiment;

FIG. 2 is a diagram illustrating a hardware configuration of the image diagnosis apparatus in the first embodiment;

FIG. 3 is a diagram illustrating an architecture of a convolutional neural network in the first embodiment;

FIG. 4 is a diagram illustrating an example of a determination result image displayed in a superimposed manner on an endoscope video in the first embodiment;

FIG. 5 is a block diagram illustrating a general configuration of an image diagnosis apparatus in a second embodiment;

FIG. 6 is a diagram illustrating an architecture of a convolutional neural network in the second embodiment;

FIGS. 7A to 7C are diagrams illustrating an example of an endoscopic image obtained by capturing an esophagus with iodine liquid scattered to the lumen of the esophagus in the second embodiment;

FIG. 8 is a diagram illustrating features of a lesion (esophageal cancer) and a subject related to an endoscope video (low speed) used for an evaluation test data set;

FIG. 9 is a diagram illustrating features of a lesion (esophageal cancer) and a subject related to an endoscope video (high speed) used for an evaluation test data set;

FIG. 10 is a diagram illustrating a comparison result of irradiation with white light and narrowband light regarding whether the presence of an esophageal cancer in an endoscope video can be properly diagnosed (sensitivity);

FIG. 11 illustrates the sensitivity, specificity, positive predictive value and negative predictive value of an image diagnosis apparatus at irradiation with white light and narrowband light;

FIGS. 12A to 12F are diagrams illustrating an example of an endoscopic image used for the evaluation test data set;

FIG. 13 is a diagram illustrating features of a subject related to the endoscopic image used for the evaluation test data set;

FIGS. 14A to 14I are diagrams illustrating various endoscopic findings in an endoscopic image;

FIG. 15 is a diagram illustrating the sensitivity, specificity, positive predictive value, negative predictive value and correct diagnosis rate of the image diagnosis apparatus and an endoscopist;

FIG. 16 is a diagram illustrating an evaluation result of the presence/absence of endoscopic findings for an endoscopic image with a multiple iodine unstained area and an evaluation result of the presence/absence of endoscopic findings for an endoscopic image with no multiple iodine unstained area;

FIG. 17 is a diagram illustrating a comparison result between the image diagnosis apparatus and the endoscopic findings regarding whether the presence of a multiple iodine unstained area in an endoscopic image can be properly diagnosed (sensitivity); and

FIG. 18 is a diagram illustrating the number of esophageal squamous cell carcinomas and head and neck squamous cell carcinomas and incidence rate per 100 person-years for a case diagnosed with an image diagnosis apparatus that a multiple iodine unstained area is present (not present) in an endoscopic image.

### DESCRIPTION OF EMBODIMENTS

The present embodiments are described below with reference to the drawings. The first embodiment includes an image diagnosis apparatus, an image diagnosis method, and an image diagnosis program with a real time video, and the second embodiment includes an image diagnosis apparatus, an image diagnosis method, and an image diagnosis program with an AI trained with training data of the multiple iodine unstained area of iodine staining of the lumen of the esophagus. In endoscope inspection of esophageal cancer, the first embodiment and the second embodiment may be implemented independently or in combination.

### General Configuration of Image Diagnosis Apparatus

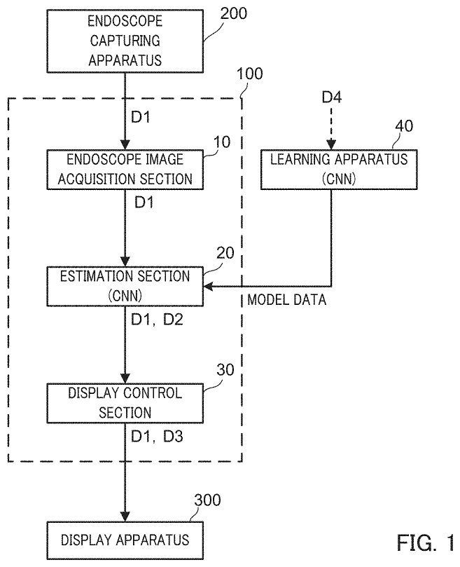

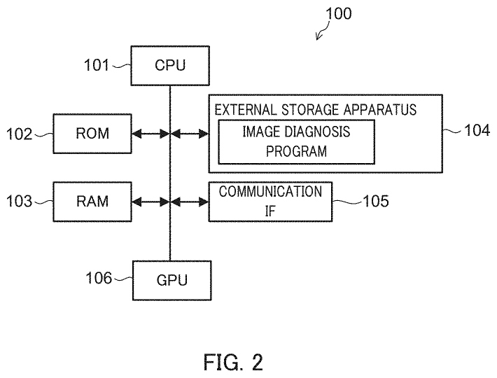

First, a configuration of image diagnosis apparatus 100 of the first embodiment (a real time video diagnosis) is described. FIG. 1 is a block diagram illustrating a general configuration of image diagnosis apparatus 100. FIG. 2 is a diagram illustrating an example of a hardware configuration of image diagnosis apparatus 100 in the first embodiment.

In endoscopy of a digestive organ (in the present embodiment, esophagus) conducted by a doctor (for example, an endoscopist), image diagnosis apparatus 100 performs diagnosis of esophageal cancer with a real time video by use of the image diagnostic capability for the endoscopic image of a convolutional neural network (CNN). Image diagnosis apparatus 100 is connected with endoscope capturing apparatus 200 and display apparatus 300.

Endoscope capturing apparatus 200 is an electronic endoscope (also referred to as video scope) with a built-in image-capturing means, a camera-equipped endoscope including an optical endoscope in which a camera head with a built-in image-capturing means is mounted or the like, for example. Endoscope capturing apparatus 200 is inserted to a digestive organ from the mouse or nose of the subject so as to capture an image of the diagnostic target portion in the digestive organ, for example.

In the present embodiment, endoscope capturing apparatus 200 captures the diagnostic target portion in the esophagus in the form of an endoscope video in the state where the esophagus of the subject is irradiated with white light or narrowband light (for example, NBI narrowband light) in accordance with the operation (for example, button operation) of the doctor. The endoscope video is composed of a plurality of temporally sequential endoscopic images. Endoscope capturing apparatus 200 outputs endoscopic image data D1 representing the captured endoscope video to image diagnosis apparatus 100.

Display apparatus 300 is, for example, a liquid crystal display, and identifiably displays, to the doctor, the determination result image and the endoscope video output from image diagnosis apparatus 100.

As illustrated in FIG. 2, image diagnosis apparatus 100 is a computer including, as main components, central processing unit (CPU) 101, read only memory (ROM) 102, random access memory (RAM) 103, external storage apparatus (for example, flash memory) 104, communication interface 105 and graphics processing unit (GPU) 106 and the like.

Each function of image diagnosis apparatus 100 is implemented with reference to the control program (such as image diagnosis program) and various data (such as endoscopic image data, learning teacher data, and the model data (such as structure data and learned weight parameter) of the convolutional neural network stored in CPU 101, GPU 106 ROM 102, RAM 103, external storage apparatus 104 and the like, for example. Note that RAM 103 functions as a working area and a temporary storage area of data, for example.

Note that a part or all of each function of image diagnosis apparatus 100 may be achieved through a process of a digital signal processor (DSP) instead of or together with the processes of CPU 101 and GPU 106. In addition, likewise, a part or all of each function may be achieved through a process of a dedicated hardware circuit instead of or together with the process of software.

As illustrated in FIG. 1, image diagnosis apparatus 100 includes endoscopic image acquisition section 10, estimation section 20 and display control section 30. Learning apparatus 40 has a function of generating the model data (corresponding to “learned model” of the present invention) of the convolutional neural network to be used in image diagnosis apparatus 100. Note that display control section 30 also functions as the “alert output control section” of the present invention.

### Endoscopic Image Acquisition Section

Endoscopic image acquisition section 10 acquires endoscopic image data D1 output from endoscope capturing apparatus 200. Then, endoscopic image acquisition section 10 outputs the acquired endoscopic image data D1 to estimation section 20. Note that when acquiring endoscopic image data D1, endoscopic image acquisition section 10 may directly acquire it from endoscope capturing apparatus 200, or may acquire endoscopic image data D1 stored in external storage apparatus 104 or endoscopic image data D1 provided through Internet connection or the like.

### Estimation Section

With the convolutional neural network, estimation section 20 estimates the presence of the lesion (in the present embodiment, esophageal cancer) in the endoscope video represented by endoscopic image data D1 output from endoscopic image acquisition section 10, and outputs the estimation result. To be more specific, estimation section 20 estimates the lesion name (name) and lesion location (position) of the lesion present in the endoscope video, and the degree of certainty (also referred to as likelihood) of the lesion name and lesion location. Then, estimation section 20 outputs, to display control section 30, endoscopic image data D1 output from endoscopic image acquisition section 10 and estimation result data D2 representing the estimation results of the lesion name, lesion location and the degree of certainty.

In addition, when a predetermined number of (for example, three) endoscopic images whose degree of certainty is greater than or equal to a predetermined value (for example, 0.5) is present in a predetermined time (for example, 0.5 seconds) in the endoscope video represented by endoscopic image data D1, estimation section 20 estimates that there is a lesion (esophageal cancer) in the endoscope video. Here, the above-mentioned predetermined number is set to decrease as the predetermined value decreases. When it is estimated that the lesion is present in the endoscope video, estimation section 20 outputs the estimation (estimation result) to display control section 30.

In the present embodiment, estimation section 20 estimates a probability score as an indicator representing the degree of certainty of the lesion name and lesion location. The probability score is represented by a value greater than 0 and equal to or smaller than 1. The higher the probability score is, the higher the degree of certainty of the lesion name and lesion location is.

Note that the probability score is an example of an indicator representing the degree of certainty of the lesion name and lesion location, and any other indicators may be used. For example, the probability score may be represented by values from 0% to 100%, or by a value of multiple-level values.

The convolutional neural network is a feedforward type of neural network, and is based on the knowledge of the structure of the visual cortex of the brain. Basically, it has a structure in which a convolutional layer responsible for extracting local features of image and a pooling layer (sub sampling layer) for collecting features for each locality are repeated. With each layer of the convolutional neural network, multiple neurons are provided, and each neuron is disposed in a manner corresponding to the visual cortex. The basic function of each neuron is composed of input of and output of signals. It should be noted that, when transmitting signals to each other, the neurons of each layer do not input the signal as it is, but sets a coupling weight to each input and outputs the signal to the neuron of the next layer when the sum of the weighted inputs exceeds the threshold value set in each neuron. The coupling weights of the neurons are calculated in advance from the learning data. In this manner, the output value can be estimated by inputting real time data. Examples of the publicly known convolutional neural network model include GoogLeNet, ResNet and SENet, but the algorithm making up the network is not limited as long as the convolutional neural network can achieve the object.

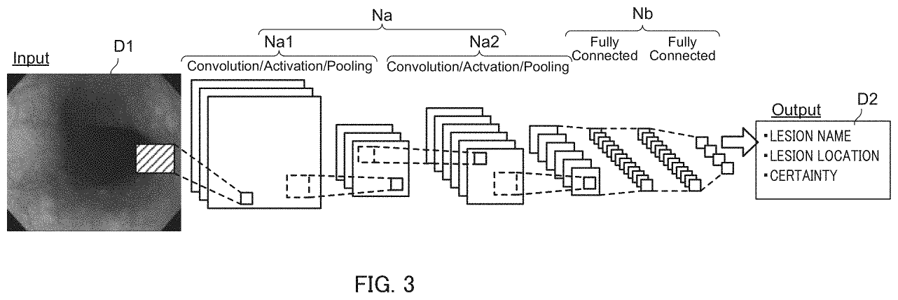

FIG. 3 is a diagram illustrating an architecture of the convolutional neural network of the present embodiment. Note that the model data (such as structure data and learned weight parameter) of the convolutional neural network is stored in external storage apparatus 104 together with an image diagnosis program.

As illustrated in FIG. 3, the convolutional neural network includes feature extraction section Na and identification section Nb, for example. Feature extraction section Na performs a process of extracting the image feature from the input image (more specifically, the endoscopic image making up the endoscope video represented by endoscopic image data D1). Identification section Nb outputs the estimation result of the image from the image feature extracted by feature extraction section Na.

Feature extraction section Na is composed of a plurality of features extraction layers Na1, Na2 . . . hierarchically connected with each other. Each of feature extraction layers Na1, Na2 . . . includes a convolutional layer, an activation layer and a pooling layer.

Feature extraction layer Na1 as the first layer scans the input image in a unit of predetermined sizes through raster scan. Then, feature extraction layer Na1 extracts the feature included in the input image by performing the feature extraction process on the scanned data with the convolutional layer, the activation layer and the pooling layer. Feature extraction layer Na1 as the first layer extracts relatively simple single features such as a linear feature extending in the horizontal direction and a linear feature extending in an oblique direction, for example. Feature extraction layer Na2 as the second layer scans an image (also called feature map) input from feature extraction layer Na1 of the previous layer in a unit of predetermined sizes through raster scan, for example. Then, feature extraction layer Na2 extracts the feature included in the input image by performing the feature extraction process on the scanned data in the same manner, with the convolutional layer, the activation layer and the pooling layer. Note that feature extraction layer Na2 as the second layer extracts a composite feature of a higher level by performing integration with reference to the positional relationship of the plurality of features extracted by feature extraction layer Na1 as the first layer and the like.

The second and subsequent feature extraction layers (FIG. 3 illustrates only two layers of feature extraction layer Na for convenience of description) execute the process as that of feature extraction layer Na2 as the second layer. Then, the output (the values of the maps of the plurality of feature maps) of the final feature extraction layer is input to identification section Nb.

Identification section Nb is composed of a multilayer perceptron where a plurality of fully connected layers are hierarchically connected, for example.

The input side fully connected layer of identification section Nb, which is fully connected to the values of the maps of the plurality of feature maps acquired from feature extraction section Na, performs sum-of-product computation on the values while changing the weight coefficient, and outputs it.

The fully connected layer of the next layer of identification section Nb, which is fully connected to the values output by elements of the fully connected layer of the previous layer, performs sum-of-product computation while applying different weight coefficients to the values. Then, at the last of identification section Nb, a layer (such as softmax function) for outputting the lesion name and lesion location of the lesion present in the image (endoscopic image) input to feature extraction section Na and the probability score (degree of certainty) of the lesion name and lesion location is provided.

The convolutional neural network may have an estimation function such that a desired estimation result (here, lesion name, lesion location and probability score) can be output from the input endoscopic image through a learning process using reference data (hereinafter referred to as “training data”) subjected beforehand to a marking process by an experienced endoscopist. At this time, through the learning with a sufficient amount of training data covering typical pathological conditions with adjusted bias and proper adjustment of weights, it is possible to prevent overfitting and produce an AI program with generalized capability for esophageal cancer diagnosis.

The convolutional neural network of the present embodiment is configured such that, with endoscopic image data D1 as an input (Input of FIG. 3), the lesion name, lesion location and probability score corresponding to the image feature of the endoscopic image making up the endoscope video represented by endoscopic image data D1 are output (Output of FIG. 3) as estimation result data D2.

Note that more preferably, the convolutional neural network may be configured to be able to input information on the age, gender, region, or past medical history of the subject (for example, may be provided as an input element of identification section Nb) in addition to endoscopic image data D1. Since the importance of the real-world data in the actual clinical practice is particularly recognized, addition of the information on the subject attributes can achieve loading in more useful systems in the actual clinical practice. Specifically, the feature of endoscopic image is considered to have correlations with the information on the age, gender, region, past medical history, family medical history and the like of the subject, and therefore, with reference to the subject's property such as the age in addition to endoscopic image data D1 for the convolutional neural network, it is possible to estimate the lesion name and lesion location with higher accuracy. This approach is a matter that should be incorporated, especially if the invention is to be utilized internationally, as the pathological condition of disease can vary by region and even between races.

In addition, estimation section 20 may perform, in addition to the process of the convolutional neural network, a process of conversion to the size and aspect ratio of the endoscopic image, a color division process of the endoscopic image, a color conversion process of the endoscopic image, a color extraction process, a luminance grade extraction process and the like as preprocessing. To prevent overfitting and increase accuracy, it is also preferable to adjust the weighting.

### Display Control Section

Display control section 30 generates a determination result image for superimposition display of the lesion name, lesion location and probability score represented by estimation result data D2 output from estimation section 20 on the endoscope video represented by endoscopic image data D1 output from estimation section 20. Then, display control section 30 outputs endoscopic image data D1 and determination result image data D3 representing the generated determination result image to display apparatus 300. In this case, digital image processing systems for image structure enhancement, color enhancement, differential processing, high contrast and high definition of the lesion of the endoscope video structure may be connected to perform processing for assisting the understanding and determination of the viewer (for example, the doctor).

Display apparatus 300 displays the determination result image represented by determination result image data D3 in a superimposed manner on the endoscope video represented by endoscopic image data D1 output from display control section 30. The endoscope video and determination result image displayed on display apparatus 300 is used for real time diagnosis assistance and diagnosis support for the doctor.

In the present embodiment, when the probability score is greater than or equal to a certain threshold value (for example, 0.4), display control section 30 displays a rectangular frame representing the lesion location, the lesion name and the probability score in a superimposed manner on the endoscope video. On the other hand, when the probability score is smaller than a certain threshold value (for example, 0.4), i.e., when the probability of the presence of a lesion in the endoscope video is low, display control section 30 does not display the rectangular frame representing the lesion location, the lesion name and the probability score on the endoscope video. That is, display control section 30 changes the display mode of the determination result image on the endoscope video in accordance with the probability score represented by estimation result data D2 output from estimation section 20.

In addition, when the estimation that the lesion is present in the endoscope video is output from estimation section 20, display control section 30 controls display apparatus 300 so as to display and output an alert by turning on the light of the display screen of the endoscope video and blinking the rectangular range of the lesion determination section. This effectively attracts the attention of the doctor to the presence of the lesion in the endoscope video. Note that when estimation section 20 estimates that the lesion is present in the endoscope video, an alert may be output by sounding (outputting) an alert sound from a speaker not illustrated in the drawing. Further, at this time, the determination probability and estimation probability may be individually calculated and displayed.

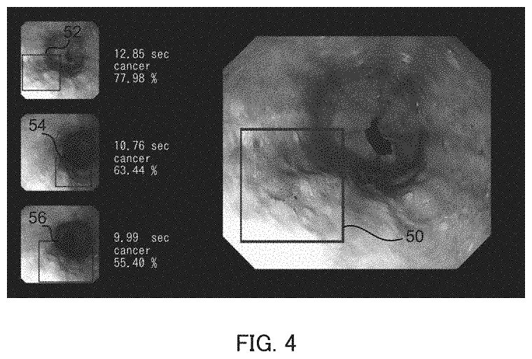

FIG. 4 is a diagram illustrating an example in which a determination result image is displayed in a superimposed manner on an endoscope video. FIG. 4 is an endoscope video obtained by capturing a diagnostic target portion in esophagus in a state where the esophagus of the subject is irradiated with narrowband light. As the endoscope video is displayed on the right side in FIG. 4, rectangular frame 50 representing the lesion location (range) estimated by estimation section 20 is displayed as a determination result image. A plurality of (for example, three) endoscopic images displayed on the left side in FIG. 4 are endoscopic images whose degree of certainty is greater than or equal to a predetermined value (for example, 0.5) displayed in the order of the capturing timing (vertical direction). As the endoscopic image is displayed on the left side in FIG. 4, rectangular frames 52, 54 and 56 representing the lesion locations (ranges) estimated by estimation section 20, the lesion name (for example, esophageal cancer: cancer) and the probability scores (for example, 77.98%, 63.44% and 55.40%) are displayed as determination result images.

### Learning Apparatus

Learning apparatus 40 performs a learning process for the convolutional neural network of learning apparatus 40 by inputting training data D4 stored in an external storage apparatus not illustrated in the drawing such that the convolutional neural network of estimation section 20 can estimate the lesion location, lesion name and probability score from endoscopic image data D1 (more specifically, the endoscopic image making up the endoscope video).

In the present embodiment, learning apparatus 40 performs a learning process by using, as training data D4, an endoscopic image (still picture image) captured with endoscope capturing apparatus 200 through irradiation of the esophaguses of a plurality of subjects with white light or narrowband light in a previously performed esophageal endoscope inspection, and the lesion name and lesion location of a lesion (esophageal cancer) present in the endoscopic image determined in advance by a doctor. To be more specific, learning apparatus 40 performs the learning process of the convolutional neural network such that errors (also called loss) of the output data for the correct value (lesion name and lesion location) obtained when the endoscopic image is input to the convolutional neural network are reduced.

In the present embodiment, learning apparatus 40 performs a learning process by using, as training data D4, the endoscopic image (corresponding to “esophageal cancer image” of the present invention) in which the lesion (esophageal cancer) is shown, i.e., present.

For the endoscopic image as teacher data D4 in the learning process, the extensive database of Japanese top-class hospital specializing in cancer treatment was mainly used, and marking of the lesion location of the lesion (esophageal cancer) was performed through specific examination, sorting, and precise manual processing on all images by a preceptor of Japan Gastroenterological Endoscopy Society with extensive diagnostic and therapeutic experience. For accuracy management and bias elimination of training data D4 (endoscopic image data) serving as reference data, a sufficient number of cases having been subjected to image sorting, lesion identification, and feature extraction marking by expert endoscopists with extensive experience are significantly important because it is directly related to the diagnosis accuracy of image diagnosis apparatus 100. With such highly accurate data cleansing operation and high quality reference data, highly reliable output results of the AI program are provided.

Training data D4 of the endoscopic image may be pixel value data, or data having been subjected to a predetermined color conversion process and the like. In addition, as preprocessing, it is also possible to use the texture feature, the shape feature, the unevenness status, the spreading feature and the like specific to cancerous areas extracted through comparison between an inflammation image and a non-inflammation image. In addition, training data D4 may be associated with information on the age, gender, region, past medical history, and family medical history of the subject and the like, in addition to the endoscopic image data to perform the learning process.

Note that the algorithm for the learning process of learning apparatus 40 may be a publicly known method. Learning apparatus 40 performs a learning process on the convolutional neural network by using, for example, publicly known backpropagation, and adjusts the network parameters (weight coefficient, bias and the like). Then, the model data (such as structure data and learned weight parameter) of the convolutional neural network having been subjected to the learning process with learning apparatus 40 is stored in external storage apparatus 104 together with the image diagnosis program, for example. Examples of the publicly known convolutional neural network model include GoogLeNet, ResNet and SENet.

As elaborated above, in the present embodiment, image diagnosis apparatus 100 includes endoscopic image acquisition section 10 that acquires an endoscope video obtained by capturing the esophagus of the subject, and estimation section 20 that estimates the presence of esophageal cancer in the endoscope video acquired by using a convolutional neural network having been subjected to learning with an esophageal cancer image obtained by capturing an esophagus where an esophageal cancer is present as training data, and outputs the estimation result.

To be more specific, the convolutional neural network has been trained based on endoscopic a plurality of images (esophageal cancer images) of esophaguses (digestive organs) obtained in advance for a plurality of subjects, and the definitive determination result of the lesion name and lesion location of the lesion (esophageal cancer) obtained in advance for each of a plurality of subjects. Thus, the lesion name and lesion location of the esophagus of a new subject can be estimated in short time with the accuracy substantially comparable to that of experienced endoscopists. Thus, in esophageal endoscope inspection, diagnosis of esophageal cancer can be performed in real time by using the diagnostic capability of the endoscope video of the convolutional neural network according to the present embodiment.

In the actual clinical practice, image diagnosis apparatus 100 may be used as a diagnosis support tool that directly supports the diagnosis of the endoscope video conducted by an endoscopist in the laboratory. In addition, image diagnosis apparatus 100 may be used as a central diagnosis support service that supports the diagnosis of endoscope videos transmitted from a plurality of laboratories, and as a diagnosis support service that supports the diagnosis of the endoscope video at remote institutions through remote control via Internet connection. In addition, image diagnosis apparatus 100 may be operated on the cloud. Further, these endoscope videos and AI determination results may be provided directly as a video library so as to be used as training materials and resources for educational training and research.

### General Configuration of Image Diagnosis Apparatus

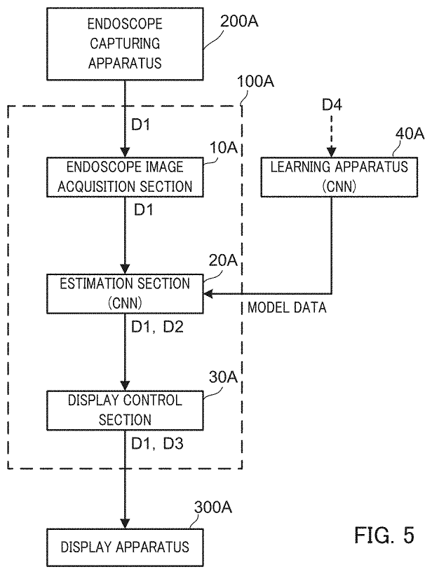

Next, a configuration of image diagnosis apparatus 100A according to a second embodiment (diagnosis through estimation of the presence or absence of multiple iodine unstained area) is described. FIG. 5 is a block diagram illustrating a general configuration of image diagnosis apparatus 100A.

Image diagnosis apparatus 100A estimates the presence or absence of the multiple iodine unstained area in the endoscopic image obtained by capturing the esophagus of the subject by using the image diagnostic capability for the endoscopic image of the convolutional neural network in endoscope inspection of a digestive organ (in the present embodiment, esophagus) conducted by a doctor (for example, an endoscopist). The multiple iodine unstained area is a portion that does not stain brown and shows yellowish-white when iodine liquid scattered to the lumen of the esophagus. Image diagnosis apparatus 100A is connected with endoscope capturing apparatus 200A and display apparatus 300A.

Endoscope capturing apparatus 200A is, for example, an electronic endoscope (also referred to as video scope) with a built-in image-capturing means, a camera-equipped endoscope including an optical endoscope in which a camera head with a built-in image-capturing means is mounted or the like. Endoscope capturing apparatus 200A is inserted to a digestive organ from the mouse or nose of the subject so as to capture an image of the diagnostic target portion in the digestive organ, for example.

In the present embodiment, endoscope capturing apparatus 200A captures, as an endoscopic image, the diagnostic target portion in the esophagus in the state where the esophagus of the subject is irradiated with white light or narrowband light (for example, NBI narrowband light) in accordance with the operation (for example, button operation) of the doctor. Endoscope capturing apparatus 200A outputs endoscopic image data D1 representing the captured endoscopic image to image diagnosis apparatus 100A.

Display apparatus 300A is, for example, a liquid crystal display, and identifiably displays, to the doctor, the endoscopic image and determination result image output from image diagnosis apparatus 100A.

As with image diagnosis apparatus 100 of the first embodiment, image diagnosis apparatus 100A is a computer including, as main components, central processing unit (CPU) 101, read only memory (ROM) 102, random access memory (RAM) 103, external storage apparatus (for example, flash memory) 104, communication interface 105 and graphics processing unit (GPU) 106 and the like (see FIG. 2).

Each function of image diagnosis apparatus 100A is implemented with reference to the control program (such as image diagnosis program) and various data (for example, endoscopic image data, training data, the model data (such as structure data and learned weight parameter) of the convolutional neural network) stored in CPU 101, GPU 106 ROM 102, RAM 103, external storage apparatus 104 and the like, for example. Note that RAM 103 functions as a working area and a temporary storage area of data, for example.

Note that a part or all of functions of image diagnosis apparatus 100A may be achieved through a process of a digital signal processor (DSP) instead of or together with the processes of CPU 101 and GPU 106. In addition, likewise, a part or all of the functions may be achieved through a process of a dedicated hardware circuit instead of or together with the process of software.

As illustrated in FIG. 5, image diagnosis apparatus 100A includes endoscopic image acquisition section 10A, estimation section 20A and display control section 30A. Learning apparatus 40A has a function of generating the model data (corresponding to “learned model” of the present invention) of the convolutional neural network used in image diagnosis apparatus 100A.

### Endoscopic Image Acquisition Section

Endoscopic image acquisition section 10A acquires endoscopic image data D1 output from endoscope capturing apparatus 200A, for example. Then, endoscopic image acquisition section 10A outputs the acquired endoscopic image data D1 to estimation section 20A. Note that when acquiring endoscopic image data D1, endoscopic image acquisition section 10A may acquire it directly from endoscope capturing apparatus 200A, or may acquire endoscopic image data D1 stored in external storage apparatus 104 or endoscopic image data D1 provided through Internet connection or the like.

### Estimation Section

With a convolutional neural network, estimation section 20A estimates the presence or absence of the multiple iodine unstained area in the endoscopic image represented by endoscopic image data D1 output from endoscopic image acquisition section 10A, and outputs the estimation result. To be more specific, estimation section 20A estimates the degree of certainty of the presence or absence (also referred to as likelihood) of the multiple iodine unstained area in the endoscopic image. Then, estimation section 20A outputs, to display control section 30A, endoscopic image data D1 output from endoscopic image acquisition section 10A and estimation result data D2 representing the estimation result of the degree of certainty of the presence or absence of the multiple iodine unstained area.

In the present embodiment, estimation section 20A estimates a probability score as an indicator representing the degree of certainty of the presence or absence of the multiple iodine unstained area. The probability score is represented by a value greater than 0 and equal to or smaller than 1. The higher the probability score is, the higher the degree of certainty of the presence or absence of the multiple iodine unstained area is.

Note that the probability score is an example of an indicator representing the degree of certainty of the presence or absence of the multiple iodine unstained area, and any other indicators may be used. For example, the probability score may be represented by values from 0% to 100%, or by a value of multiple-level values.

Convolutional neural network is a feedforward type of neural network, and is based on the knowledge of the structure of the visual cortex of the brain. Basically, it has a structure in which a convolutional layer responsible for extracting local features of image and a pooling layer (subsampling layer) for collecting features for each locality are repeated. With each layer of the convolutional neural network, a plurality of neurons is provided, and each neuron is disposed in a manner corresponding to the visual cortex. The basic function of each neuron is composed of input of and output of signals.

It should be noted that, when transmitting signals to each other, the neurons of each layer do not input the signal as it is, but sets a coupling weight to each input and outputs the signal to the neuron of the next layer when the sum of the weighted inputs exceeds the threshold value set in each neuron. The coupling weights of the neurons are calculated in advance from the learning data. In this manner, the output value can be estimated by inputting real time data. But the algorithm making up the network is not limited as long as the convolutional neural network can achieve the object.

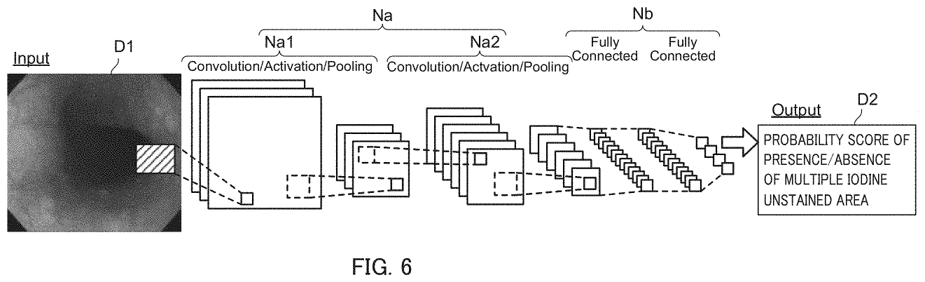

FIG. 6 is a diagram illustrating an architecture of the convolutional neural network of the present embodiment. Note that the model data (such as structure data and learned weight parameter) of the convolutional neural network is stored in external storage apparatus 104 together with the image diagnosis program.

As illustrated in FIG. 6, the convolutional neural network includes feature extraction section Na and identification section Nb, for example. Feature extraction section Na performs a process of extracting the image feature from the input image (more specifically, endoscopic image represented by endoscopic image data D1). Identification section Nb outputs the estimation result of the image from the image feature extracted by feature extraction section Na.

Feature extraction section Na is composed of a plurality of features extraction layers Na1, Na2 . . . hierarchically connected with each other. Each of feature extraction layers Na1, Na2 . . . includes a convolutional layer, an activation layer and a pooling layer.

Feature extraction layer Na1 as the first layer scans the input image in a unit of predetermined sizes through raster scan. Then, feature extraction layer Na1 extracts the feature included in the input image by performing the feature extraction process on the scanned data with the convolutional layer, the activation layer and the pooling layer. Feature extraction layer Na1 as the first layer extracts relatively simple single features such as a linear feature extending in the horizontal direction and a linear feature extending in an oblique direction, for example.

Feature extraction layer Na2 as the second layer scans an image (also called feature map) input from feature extraction layer Na1 of the previous layer in a unit of predetermined sizes through raster scan, for example. Then, feature extraction layer Na2 extracts the feature included in the input image by performing the feature extraction process on the scanned data in the same manner, with the convolutional layer, the activation layer and the pooling layer. Note that feature extraction layer Na2 as the second layer extracts a composite feature of a higher level by performing integration with reference to the positional relationship of the plurality of features extracted by feature extraction layer Na1 as the first layer and the like.

The second and subsequent feature extraction layers (in FIG. 6, only two layers of feature extraction layer Na for convenience of description) execute the process as that of feature extraction layer Na2 as the second layer. Then, the output (the values of the maps of the plurality of feature maps) of the final feature extraction layer is input to identification section Nb.

Identification section Nb is composed of a multilayer perceptron where a plurality of fully connected layers are hierarchically connected, for example.

The input side fully connected layer of identification section Nb, which is fully connected to the values of the maps of the plurality of feature maps acquired from feature extraction section Na, performs sum-of-product computation on the values while changing the weight coefficient, and outputs it.

The fully connected layer of the next layer of identification section Nb, which is fully connected to the values output by elements of the fully connected layer of the previous layer, performs sum-of-product computation while applying different weight coefficients to the values. Then, at the last of identification section Nb, a layer (such as softmax function) for outputting the probability score (degree of certainty) of the presence or absence of the multiple iodine unstained area in the image (endoscopic image) input to feature extraction section Na is provided.

The convolutional neural network may have an estimation function such that a desired estimation result (here, the probability score of the presence or absence of the multiple iodine unstained area) can be output from the input endoscopic image through a preliminary learning process using reference data (hereinafter referred to as “training data”) subjected beforehand to a marking process by an experienced endoscopist. At this time, through the learning with a sufficient amount of training data covering typical pathological conditions with adjusted bias and proper adjustment of weights, overfitting can be prevented. In addition, by connecting an AI program with generalized capability for diagnosis of the presence or absence of the multiple iodine unstained area of the present embodiment, a program with diagnostic capability with high speed and high accuracy can be achieved.

The convolutional neural network of the present embodiment is configured such that with endoscopic image data D1 as an input (Input of FIG. 6), it outputs, as estimation result data D2 (Output of FIG. 6), the probability score of the presence or absence of the multiple iodine unstained area according to the image feature of the endoscopic image represented by endoscopic image data D1.

Note that more preferably, the convolutional neural network may have a configuration in which information on the age, gender, region or past medical history of the subject, in addition to endoscopic image data D1, can be input (for example, it is provided as an input element of identification section Nb). Since the importance of the real-world data in the actual clinical practice is particularly recognized, addition of the information on the subject attributes can achieve loading in more useful systems in the actual clinical practice. Specifically, the feature of endoscopic image is considered to have correlations with the information on the age, gender, region, past medical history, family medical history and the like of the subject, and therefore, with reference to the subject's property such as the age in addition to endoscopic image data D1 for the convolutional neural network, the presence or absence of the multiple iodine unstained area can be estimated with higher accuracy. This approach is a matter that should be incorporated, especially if the invention is to be utilized internationally, as the pathological condition of disease can vary by region and even between races.

In addition, estimation section 20A may perform, in addition to the process of the convolutional neural network, a process of conversion to the size and aspect ratio of the endoscopic image, a color division process of the endoscopic image, a color conversion process of the endoscopic image, a color extraction process, a luminance grade extraction process and the like as preprocessing. Note that to prevent overfitting and increase accuracy, it is also preferable to adjust the weighting.

### Display Control Section

Display control section 30A generates a determination result image for superimposition display of the probability score represented by estimation result data D2 output from estimation section 20A on endoscope image represented by endoscopic image data D1 output from estimation section 20A. Then, display control section 30A outputs endoscopic image data D1 and determination result image data D3 representing the generated determination result image to display apparatus 300A. In this case, digital image processing systems for image structure enhancement, color enhancement, differential processing, high contrast and high definition of the endoscope image structure may be connected to perform processing for assisting the understanding and determination of the viewer (for example, the doctor).

Display apparatus 300A displays the determination result image represented by determination result image data D3 in a superimposed manner on the endoscope image represented by endoscopic image data D1 output from display control section 30A. The endoscope image and determination result image displayed on display apparatus 300A is used for real time diagnosis assistance and diagnosis support for the doctor.

In the present embodiment, when the probability score is greater than or equal to a certain threshold value (for example, 0.6), display control section 30A controls display apparatus 300A to turn on the light of a screen displaying the endoscopic image and thus can output an alert of the presence of a multiple iodine unstained area. This effectively attracts the attention of the doctor to the presence of the multiple iodine unstained area in the endoscope image. Note that when the probability score is greater than or equal to a certain threshold value, image diagnosis apparatus 100A may output an alert by sounding (outputting) an alert sound from a speaker not illustrated in the drawing. Further, at this time, the determination probability and estimation probability may be individually calculated and displayed.

### Learning Apparatus

Learning apparatus 40A performs a learning process for the convolutional neural network of learning apparatus 40A by inputting training data D4 stored in an external storage apparatus not illustrated in the drawing such that the convolutional neural network of estimation section 20A can estimate the probability score of the presence or absence of the multiple iodine unstained area from endoscopic image data D1 (more specifically, the endoscopic image).

In the present embodiment, learning apparatus 40A performs a learning process by using, as training data D4, an endoscopic image captured with endoscope capturing apparatus 200A with irradiation of the esophaguses of a plurality of subjects with white light or narrowband light in a previously performed esophageal endoscopy, and the presence or absence of the multiple iodine unstained area in the endoscopic image determined in advance through iodine staining for confirmation. To be more specific, learning apparatus 40A performs the learning process of the convolutional neural network such that errors (also called loss) of the output data for the correct value (the presence or absence of the multiple iodine unstained area) obtained when the endoscopic image is input to the convolutional neural network are reduced.

In the present embodiment, learning apparatus 40A performs a learning process by using, as training data D4, an endoscopic image obtained by actually capturing the esophagus where a multiple iodine unstained area is present (corresponding to “unstained area image” of the present invention), and an endoscopic image obtained by actually capturing the esophagus where no multiple iodine unstained area is not present (corresponding to “non-unstained area image” of the present invention).

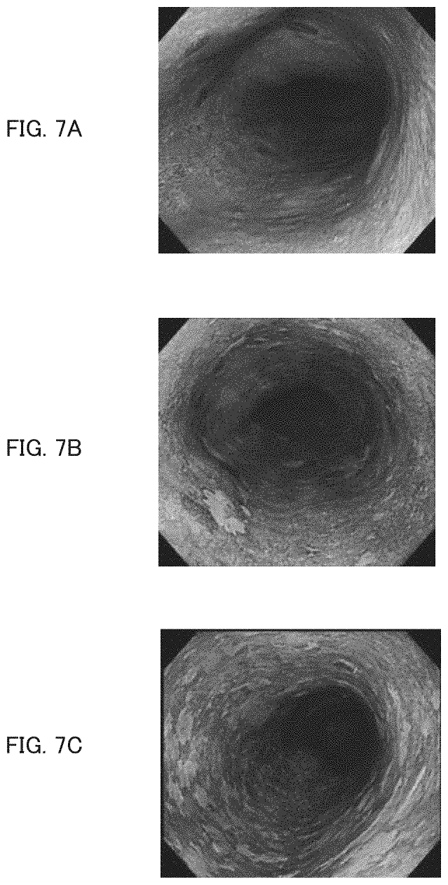

FIG. 7 is a diagram illustrating an example of an endoscopic image obtained by capturing an esophagus with iodine liquid scattered to the lumen of the esophagus. In the endoscopic image illustrated in FIG. 7A, the number of the multiple iodine unstained areas present in the esophagus is 0, and the doctor determines that there is no multiple iodine unstained area in the endoscopic image (grade A). In the endoscopic image illustrated in FIG. 7B, the number of the multiple iodine unstained areas present in the esophagus is 1 to 9, and the doctor determines that there is no multiple iodine unstained area in the endoscopic image (grade B). In the endoscopic image illustrated in FIG. 7C, the number of the multiple iodine unstained areas present in the esophagus is 10 or greater, and the doctor determines that there is the multiple iodine unstained area in the endoscopic image (grade C). The endoscopic image processing device (image diagnosis apparatus 100A) driven with the program learned with that training data of the multiple iodine unstained area can estimate the multiple iodine unstained area without performing the iodine staining.

For the endoscopic image as teacher data D4 in the learning process, the extensive database of Japanese top-class hospital specializing in cancer treatment was mainly used, and all endoscopic images were specifically examined by preceptors of Japan Gastroenterological Endoscopy Society with extensive diagnostic and therapeutic experience to determine the presence or absence of the multiple iodine unstained area. For accuracy management and bias elimination of training data D4 (endoscopic image data) serving as reference data, sufficient number of cases having been subjected to determination of the presence or absence of the multiple iodine unstained area and image sorting by expert endoscopists with extensive experience are significantly important steps because it is directly related to the diagnosis accuracy of image diagnosis apparatus 100A. With such highly accurate data cleansing operation and high quality reference data, highly reliable output results of the AI program are provided.

Training data D4 of the endoscopic image may be pixel value data, or data having been subjected to a predetermined color conversion process and the like. In addition, it is possible to use the texture feature, the shape feature, the unevenness status, the spreading feature and the like specific to the presence or absence of the multiple iodine unstained area extracted through comparison between unstained area image and non-unstained area image as preprocessing. In addition, training data D4 may be associated with information on the age, gender, region, past medical history, and family medical history of the subject and the like, in addition to the endoscopic image data to perform the learning process.

Note that the algorithm for the learning process of learning apparatus 40A may be a publicly known method. Learning apparatus 40A performs a learning process on the convolutional neural network by using, for example, publicly known backpropagation, and adjusts the network parameters (weight coefficient, bias and the like). Then, the model data (such as structure data and learned weight parameter) of the convolutional neural network having been subjected to the learning process with learning apparatus 40A is stored in external storage apparatus 104 together with the image diagnosis program, for example. Examples of the publicly known convolutional neural network model include GoogleNet, ResNet and SENet.

As elaborated above, in the present embodiment, image diagnosis apparatus 100A includes endoscopic image acquisition section 10A that acquires an endoscopic image obtained by capturing the esophagus of the subject, estimation section 20A that uses a convolutional neural network subjected to learning with a multiple iodine unstained area esophagus image obtained by capturing the esophagus where a multiple iodine unstained area is present and a non-multiple iodine unstained area esophagus image obtained by capturing the esophagus where no multiple iodine unstained area is present as training data and configured to detect a multiple iodine unstained area without performing iodine staining, to estimate the presence or absence of the multiple iodine unstained area in the acquired endoscopic image and output the estimation result. The presence of the multiple iodine unstained area leads to high cancer risk, and therefore image diagnosis apparatus 100A of the present embodiment can be used for diagnosis with the esophageal cancer risk determination function as it is.

To be more specific, the convolutional neural network has been trained based on a plurality of endoscopic images (multiple iodine unstained area esophagus images and non-multiple iodine unstained area esophagus images) of esophaguses (digestive organs) obtained in advance for a plurality of subjects, and the definitive determination result of the presence or absence of the multiple iodine unstained area obtained in advance for each of a plurality of subjects. In this manner, the presence or absence of the multiple iodine unstained area in the endoscopic image obtained by capturing the esophagus of a new subject can be estimated. Thus, in a typical endoscope inspection using no iodine staining, diagnosis can be conducted while estimating the presence or absence of the multiple iodine unstained area, which is an indicator of the esophageal cancer high-risk case, by using the diagnostic capability of the endoscopic image of the convolutional neural network according to the present embodiment. As a result, as with the case where iodine staining is performed in advance, the esophageal cancer high-risk case can be identified in advance and the esophageal cancer can be highly accurately and efficiently detected without giving the physical load of iodine staining to the subject, and, the determination of the presence/absence of the esophageal cancer using a real time video can be efficiently performed by predicting the presence of the multiple iodine unstained area by using AI without performing iodine staining together with the diagnosis using a real time video according to the first embodiment of the present invention.

In the actual clinical practice, image diagnosis apparatus 100A may be used as a diagnosis support tool that directly supports the diagnosis of the endoscope image conducted by an endoscopist in the laboratory. In addition, image diagnosis apparatus 100A may be used as a central diagnosis support service that supports the diagnosis of endoscope images transmitted from a plurality of laboratories, and as a diagnosis support service that supports the diagnosis of the endoscope image at remote institutions through remote control via Internet connection. In addition, image diagnosis apparatus 100A may be operated on the cloud. Further, these endoscope images and AI determination results may be provided directly as a video library so as to be used as teaching materials and resources for educational training and research.

Together with the cancer risk evaluation through the estimation determination of the multiple iodine unstained area, it is possible to easily perform the diagnosis with higher efficiency and accuracy with the function of optimizing the operation of the operator such as low speed observation for high risk cases and high speed observation for low risk cases through a method of determining low speed mode or high speed mode at the time of endoscope insertion. Specifically, when an endoscope is inserted to the esophagus, the degree of the esophageal cancer risk can be determined first from the detection status of the multiple iodine unstained area, and, on the basis of the determination, the setting of endoscope reference insertion speed and the alert sensitivity can be indicated on the image device display section so as to reset the operation condition such that the diagnosis can be performed under a condition suitable for the observation of the lumen of the esophagus. Regarding the endoscope insertion speed during the inspection, an alert can be output such that the difference between the reference insertion speed and the actual insertion speed is small, and thus the proper observation condition can be maintained. When no multiple iodine unstained area is detected and the cancer risk is low, it can quickly pass through the lumen of the esophagus, but in such a case, the focus that is difficult for the endoscopist to detect can be adequately detected with a real-time image diagnosis apparatus. On the other hand, when a multiple iodine unstained area is detected and the cancer risk is high, the endoscopist observes it in detail, and it is thus possible to perform precise diagnosis that does not miss microscopic cancer lesions by the endoscopist and the real-time image diagnosis apparatus together. In this manner, through the combination of the endoscope real time video diagnosis and the estimation determination of multiple iodine unstained area, the degree of the risk of the esophageal cancer can be immediately determined by only inserting the endoscope into the esophagus without capturing still pictures or performing iodine staining, and the esophageal cancer risk can be efficiently determined at a rate far beyond the speed of human judgment by complementing and extending the human determination, in which the accuracy of observation of affected areas is low with fast movement and high with slow movement. In this manner, the subject can undergo the inspection with the shortest time and the least amount of physical strain.

In this manner, with an appropriate combination of the first embodiment (diagnosis with an endoscope with a real time video) and the second embodiment (estimation determination of multiple iodine unstained area), it is possible to adjust the endoscope reference insertion speed that enables observation in accordance with the cancer risk of each subject, and the diagnosis of esophageal cancer can be more efficiently and accurately assisted than known technology.

The first and second embodiments described above are merely examples of embodiments for implementing the invention, and the technical scope of the invention should not be interpreted as limited by these embodiments. In other words, the invention can be implemented in various forms without departing from its gist or its main features.

Finally, evaluation tests for confirming the effects of the configurations of the first and second embodiments described above are described below.

### First Evaluation Test

First, a first evaluation test (endoscope real time video determination) for confirming the effects of the configuration of the first embodiment described above is described.

### Preparation of Training Data Set

8428 endoscopic still images of 429 lesions histologically diagnosed as esophageal cancer from 2014 to 2017 were prepared as a training data set (training data) used for the learning of the convolutional neural network in the image diagnosis apparatus. As the endoscope capturing apparatus, GIF-H240Z, GIF-H260Z and GIF-H290 available from Olympus Medical Systems Corp were used.

Note that the endoscopic images as the training data set include endoscopic images in which an esophageal cancer is recognized (present) in the image among endoscopic images obtained by capturing the esophagus of the subject with an endoscope capturing apparatus. On the other hand, endoscopic images whose image quality is poor due to mucus and blood adhering in a wide area, out of focus or halation were excluded from the training data set. A Japan Gastroenterological Endoscopy Society preceptor, a specialist in esophageal cancer, prepared the training data set by specifically examining and sorting the prepared endoscopic images and performing marking of lesion locations through precise manual processing.

### Learning and Algorithm

For construction of the image diagnosis apparatus for the diagnosis of esophageal cancer, GoogleNet composed of 22 layers with sufficient number of parameters and expressive power and with common structure with previous convolutional neural networks was used as the convolutional neural network. Caffedeep learning framework developed at Berkeley Vision and Learning Center (BVLC) was used for the learning and evaluation test. All layers of the convolutional neural network were fine-tuned using stochastic gradient descent with a global learning rate of 0.0001. For the compatibility with convolutional neural network, each endoscopic image was resized to 224×224 pixels.

### Preparation of Evaluation Test Data Set

To evaluate the diagnosis accuracy of the image diagnosis apparatus of the constructed convolutional neural network base, in cases in which ESD was performed as initial treatment in the Cancer Institute Hospital of JFCR from August 2018 to August 2019, first, one set of 32 endoscope close inspection videos obtained by capturing the esophaguses of a plurality of subjects with an endoscope capturing apparatus, including observations of white light and narrowband light for the esophaguses of the plurality of subjects having esophageal cancer, a total of 40 endoscope videos of white light and narrowband light in 20 cases where esophageal cancer is present in normal inspection videos captured with an endoscope capturing apparatus with irradiation of the esophaguses of the plurality of subjects with white light or narrowband light, and a total of 40 endoscope videos of white light or narrowband light in 20 cases where no esophageal cancer not is present obtained by capturing the esophaguses of the plurality of subjects with an endoscope capturing apparatus, were collected as the evaluation test data set. An endoscope video in which an esophageal cancer is is shown, and an endoscope video in which no esophageal cancer is shown were captured. The frame rate of each endoscope video making up the evaluation test data set is 30 fps (one endoscopic image=0.033 seconds). As with the preparation of the teacher data set, GIF-H240Z, GIF-H260Z and GIF-H290 available from Olympus Medical Systems Corp were used as the endoscope capturing apparatus. For the structure emphasis at the time of capturing, A-mode level 5 was set for the case of white light irradiation, and B-mode level 8 was set for the case of narrowband light irradiation.

Note that the evaluation test data set includes, as the endoscope video that meets eligibility criteria, an endoscope video captured for five seconds with an endoscope capturing apparatus in a state of focusing on the esophagus of the subject as a close inspection video. In addition, as a normal inspection video (more specifically, a video showing detailed observation for close examination of lesions), an endoscope video (low speed) of a lesion observed with a movement of the endoscope at a low speed (for example, 1 cm/s) was captured. In addition, as a normal inspection video, an endoscope video (high speed) obtained by quickly inserting the endoscope at a high speed (for example, 2 cm/s) from the esophagus inlet to the esophagogastric junction was captured. On the other hand, the endoscope videos whose image quality is poor due to mucus and blood adhering in a wide area, out of focus or halation were excluded from the evaluation test data set as endoscope videos that meets the exclusion criteria. A Japan Gastroenterological Endoscopy Society preceptor, a specialist in esophageal cancer, prepared the evaluation test data set by specifically examining the prepared endoscope videos and sorting the endoscope video where esophageal cancer is present and the endoscope video where esophageal cancer is not present.

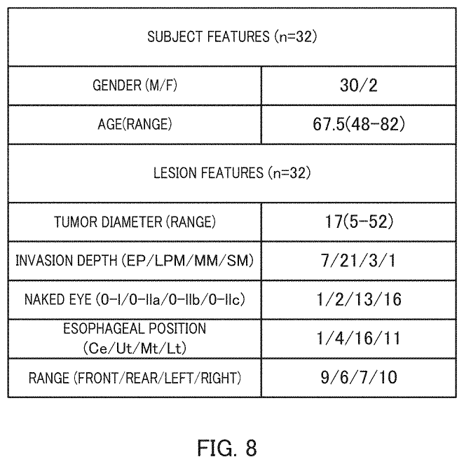

FIG. 8 is a diagram illustrating features of a lesion (esophageal cancer) and a subject related to the endoscope video (low speed) used for the evaluation test data set. For the age and the tumor diameter, center values (entire ranges) are shown. As illustrated in FIG. 8, for example, the center value of the tumor diameter was 17 mm. In terms of the depth of invasion, results were seven lesions in the mucosal epithelium (EP), 21 lesions in the mucosal lamina propria (LPM), three cases in the muscularis mucosae (MM), and one lesion in the submucosa (SM). With the naked eye (classification), the depression type (0-llc) was the largest, with 16 lesions.

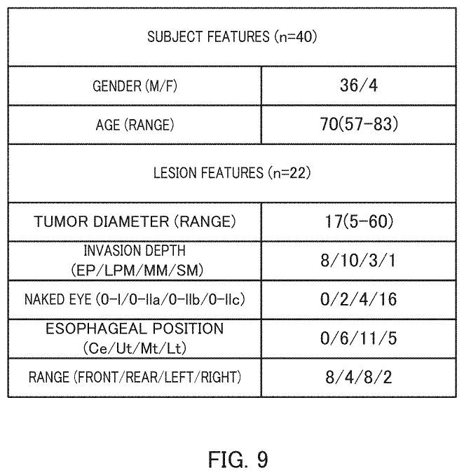

FIG. 9 is a diagram illustrating features of a lesion (esophageal cancer) and a subject related to the endoscope video (high speed) used for the evaluation test data set. For the age and the tumor diameter, center values (entire ranges) are shown. As illustrated in FIG. 8, for example, the center value of the tumor diameter was 17 mm. In terms of the depth of invasion, eight lesions in the mucosal epithelium (EP), 10 lesions in the mucosal lamina propria (LPM), three cases in the muscularis mucosae (MM), and one lesion in the submucosa (SM). With the naked eye (classification), the depression type (0-llc) was the largest, with 16 lesions.

### Method of Evaluation Test

In the present evaluation test, the evaluation test data set was input to the image diagnosis apparatus of the convolutional neural network base having been subjected to a learning process using the training data set, and whether the esophageal cancer is present in each endoscope video making up the evaluation test data set can be properly diagnosed was evaluated. When there are a predetermined number of endoscopic images whose degree of certainty is greater than or equal to a predetermined value within a predetermined time, the image diagnosis apparatus diagnoses that the lesion is present in the endoscope video.

To be more specific, the image diagnosis apparatus recognizes an endoscope video of one second as still-picture images of 30 frames. When the image diagnosis apparatus recognizes that there is an esophageal cancer, it returns back 0.5 seconds (15 frames) and performs the searching, and, if there are three or more frames of endoscopic images including esophageal cancer, it diagnoses that there is an esophageal cancer in the endoscope video.

In addition, in the present evaluation test, the presence of an esophageal cancer can be properly diagnosed by the image diagnosis apparatus (sensitivity) in endoscope videos captured in the state where the esophagus of the subject is irradiated with white light and narrowband light was calculated by using the following expression (1).

Sensitivity=(the number of endoscope videos that have been properly diagnosed about the presence of the esophageal cancer in the evaluation test data set)/(the number of endoscope videos where esophageal cancer is actually present in the evaluation test data set) (1)

In addition, in the present evaluation test, in endoscope videos captured in the state where the esophagus of the subject is irradiated with white light and narrowband light, the specificity, positive predictive value (PPV) and negative predictive value (NPV) with respect to the diagnostic capability of the image diagnosis apparatus were calculated by using the following expressions (2) to (4).

Specificity=(the number of endoscope videos that have been properly diagnosed that there is no esophageal cancer in the evaluation test data set)/(the number of endoscope videos where esophageal cancer is actually not present in the evaluation test data set) (2)

Positive predictive value (PPV)=(the number of endoscope videos where esophageal cancer is actually present among endoscope videos diagnosed that there is esophageal cancer is present in the evaluation test data set)/(the number of endoscope videos diagnosed that there is an esophageal cancer in the evaluation test data set) (3)

Negative predictive value (NPV)=(the number of endoscope videos where esophageal cancer is actually not present among endoscope videos diagnosed that there is no esophageal cancer in the evaluation test data set)/(the number of endoscope videos that are diagnosed that there is an esophageal cancer in the evaluation test data set) (4)

### Result of Evaluation Test

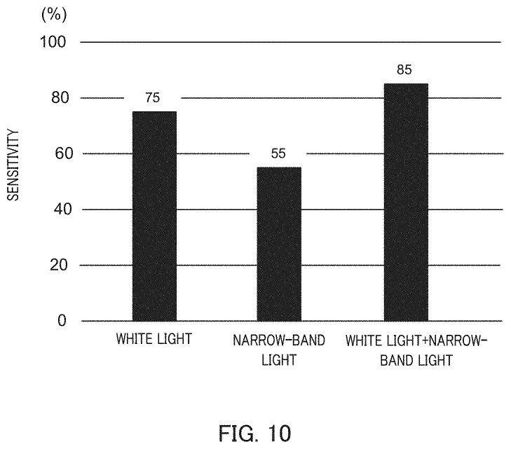

FIG. 10 is a diagram illustrating the sensitivity of the image diagnosis apparatus in an endoscope video captured in the state where the esophagus of the subject is irradiated with white light and narrowband light. As illustrated in FIG. 10, the image diagnosis apparatus properly diagnosed that there is an esophageal cancer regarding endoscope videos of 75% (95% CI) of endoscope videos captured in the state where the esophagus of the subject is irradiated with white light. In addition, the image diagnosis apparatus properly diagnosed that there is an esophageal cancer regarding the endoscope videos of 55% (95% CI) of endoscope videos captured in the state where the esophagus of the subject is irradiated with narrowband light. In addition, the image diagnosis apparatus properly diagnosed that there is an esophageal cancer regarding endoscope videos of 85% (95% CI) of endoscope videos captured in the state where the esophagus of the subject is irradiated with white light or narrowband light.

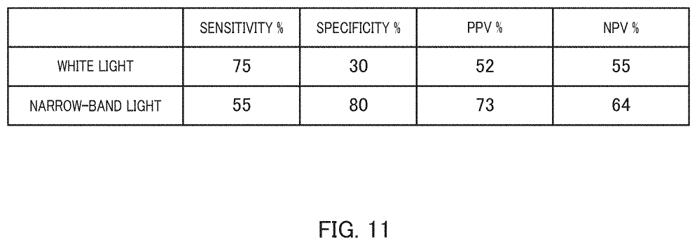

FIG. 11 is a diagram illustrating the sensitivity, specificity, positive predictive value (PPV) and negative predictive value (NPV) with respect to the diagnostic capability of the image diagnosis apparatus in endoscope videos captured in the state where the esophagus of the subject is irradiated with white light and narrowband light. As illustrated in FIG. 11, in endoscope videos captured in the state where the esophagus of the subject is irradiated with white light, the sensitivity, specificity, positive predictive value and negative predictive value of the image diagnosis apparatus were 75%, 30%, 52% and 55%, respectively. In addition, in the endoscope videos captured in the state where the esophagus of the subject is irradiated with narrowband light, the sensitivity, specificity, positive predictive value and negative predictive value of the image diagnosis apparatus were 55%, 80%, 73% and 64%, respectively.

### Considerations for First Evaluation Test

In (32) endoscope videos observed in detail for close examination of lesions, the image diagnosis apparatus has successfully recognize all esophageal cancers with both white light and narrowband light. Next, in endoscope videos obtained with quick insertion at 2.0 cm/s from the esophagus inlet to the esophagogastric junction in which whether the presence of esophageal cancer is unknown, the image diagnosis apparatus was able to recognize 85% esophageal cancers when both the white light and narrowband light were added. Diagnosis of similar quick endoscope videos by 15 endoscopists (seven medical specialists certified by Japan Gastroenterological Endoscopy Society and eight non-medical specialists who have diagnosed esophageal cancer in actual clinical practice) resulted in a correct diagnosis rate of a center value of 45% (25-60%). In addition, in the case of AI-assist endoscope videos in which the region recognized to be esophageal cancer by the image diagnosis apparatus is indicated with a square frame, the correct diagnosis rate was increased by a center value of 10% (5-20%) in 11 of 15 endoscopists.

From the above, when the endoscope insertion speed is as slow as about 1.0 cm/s, it is recognized that both the AI and endoscopists can diagnose substantially all esophageal cancers. However, when the insertion speed is about 2.0 cm/s, it is extremely difficult for endoscopists to recognize the lesion. With AI indicating the position of esophageal cancer with the square frame, the recognition of the endoscopist of the lesion was slightly improved. Conversely, AI can pick up the esophageal cancer with a high accuracy to a certain degree.

NPL 3 discloses evaluation results regarding the diagnostic capability of a computer-aided diagnosis (CAD) system for the esophageal cancer using an endoscopic image (still picture image) captured with an NBI combined magnifying endoscope were a sensitivity of 77%, a specificity of 79%, a positive predictive value of 39%, and a negative predictive value of 95%. In addition, it discloses that examples of false positive case include severe shadows, normal structures (esophagogastric junction, left main vascular branch, vertebral bod), and benign lesions (scar, local atrophy, Barrett's esophagus).

In NPL 3, however, the diagnostic capability of the computer-aided diagnosis system and the diagnostic capability of a skilled endoscopist who have mastered diagnosis techniques of esophageal cancer are not compared with each other, and therefore the difficulty of the endoscopic image diagnosis used for evaluating the diagnostic capability is unknown, thus limiting the interpretation of the diagnostic capability of the computer-aided diagnosis system.

In addition, in NPL 3, consideration is made using a still picture image (endoscopic image), which is useful for the case where secondary reading of the endoscopic image is performed after the endoscope inspection; however, it is difficult to be introduced to the actual medical field where diagnosis of esophageal cancer is performed in real time because consideration using videos is not performed. To apply it to real time videos, reconfiguration and optimization of AI algorithm are additionally required.

As described above, in the known preceding technique, which does not take into account a real time video, the evaluation of the usability and accuracy in the actual clinical practice is insufficient and the industrial usability is also limited in comparison with the present invention. In contrast, the present invention achieves the means for solving the problems, and is superior to the known technology in the following points.

(1) In the image diagnosis apparatus in the present invention, the diagnostic capability is compared with many endoscopists, and therefore the weighting and the parameter setting in the convolutional neural network are appropriate, and further, the difficulty of the video evaluation can be properly evaluated. Through the comparison with many endoscopists, it is also possible to adjust the reduction of the bias caused in the comparison with a small number of endoscopists. On top of that, the CAD system can provide the performance with the diagnostic capability comparable to or greater than skilled medical practitioners. Applicability as education and training system is proved in addition to the utilization in the actual clinical practice.
(2) In the present invention, since normally an endoscope and an NBI combined with non-magnifying endoscope are used, the diagnostic capability is high, and thus the usability in the actual clinical practice is high.
(3) In the present invention, videos are used instead of still picture images, and thus endoscopic diagnosis of the esophageal cancer can be performed in real time by using the image diagnosis apparatus in the actual clinical practice. In this manner, the task and time for recheck and determination after the inspection of the still picture image is eliminated, and the diagnosis of esophageal cancer can be immediately supported at the time of endoscope inspection, thus achieving high excellency in terms of inspection efficiency and cost effectiveness.
(4) In the diagnosis with still picture images, the evaluation is made for captured photographs alone, and consequently the number of esophageal cancers detected at the time of endoscope inspection is limited. With the video in the present invention, the lumen of the esophagus can be successively observed regardless of the timing of capturing the affected area unlike still picture images, and thus the esophageal cancer can be detected in real time during the inspection and the number of detectable esophageal cancers is not limited, which are very useful in terms of the surveillance of an esophageal cancer in the actual clinical practice.

### Second Evaluation Test

Next, a second evaluation test (determination of multiple iodine unstained area) for confirming the effect of the configuration of the above-described second embodiment is described.

### Preparation of Teacher Data Set

In daily clinical practice in the Cancer Institute Hospital of JFCR conducted from April 2015 to October 2018, for cases where iodine staining was performed, an endoscopic image captured with an endoscope capturing apparatus in the state where the esophaguses of a plurality of subjects are irradiated with white light or narrowband light was extracted from an electronic medical record apparatus. Then, the extracted endoscopic images were prepared as a training data set (training data) used for the learning of the convolutional neural network in the image diagnosis apparatus. The breakdown is 2736 endoscopic images (white light observation: 1294 and narrowband light observation: 1442) in 188 cases where a multiple iodine unstained area is present in the esophagus, and 3898 endoscopic images (white light observation: 1954 and narrowband light observation: 1944) in 407 cases where a multiple iodine unstained area is actually not present in the esophagus. As the endoscope capturing apparatus, a high resolution endoscope (GIF-H290Z, Olympus Medical Systems Corp, Tokyo) and a high resolution endoscope video system (EVIS LUCERA ELITE CV-290/CLV-290SL, Olympus Medical Systems Corp, Tokyo) were used. Regarding the structure emphasis at the time of capturing, A-mode level 5 was set for the case of white light irradiation, and B-mode level 8 was set for the case of narrowband light irradiation.

Note that endoscopic images captured for a case with a history of esophagectomy and endoscopic images captured for a case with chemotherapy and radiation therapy to the esophagus were excluded from the training data set. In addition, endoscopic images with poor image quality due to Poor air delivery, bleeding after biopsy, halos, blurring, defocusing, and mucus, and endoscopic images including esophageal cancer were also excluded from the training data set. (Two) preceptors of Japan Gastroenterological Endoscopy Society with extensive diagnostic and therapeutic experience specifically examined the prepared endoscopic images, determined the presence or absence of the multiple iodine unstained area, and prepared a training data set.

### Learning and Algorithm

To construct the image diagnosis apparatus that estimates the presence or absence of the multiple iodine unstained area in the endoscopic image obtained by capturing the esophagus of the subject, GoogleNet composed of 22 layers with sufficient number of parameters and expressive power and with common structure with previous convolutional neural networks was used as a convolutional neural network. Caffedeep learning framework developed at Berkeley Vision and Learning Center (BVLC) was used for the learning and evaluation test. All layers of the convolutional neural network were fine-tuned using stochastic gradient descent with a global learning rate of 0.0001. For the compatibility with convolutional neural network, each endoscopic image was resized to 224×224 pixels.

### Preparation of Evaluation Test Data Set

To evaluate the diagnosis accuracy of the image diagnosis apparatus of the constructed convolutional neural network base, for cases where iodine staining was performed in daily clinical practice in the Cancer Institute Hospital of JFCR conducted from November 2018 to July 2019, endoscopic images captured with an endoscope capturing apparatus in the state where the esophaguses of a plurality of subjects are irradiated with white light or narrowband light were collected as the evaluation test data set. The breakdown is: 342 endoscopic images (white light observation: 135 and narrowband light observation: 207) in 32 cases where a multiple iodine unstained area is actually present in the esophagus, and 325 endoscopic images (white light observation: 165 and narrowband light observation: 160) in 40 cases where a multiple iodine unstained area is actually not present in the esophagus. As the endoscope capturing apparatus, a high resolution endoscope (GIF-H290Z, Olympus Medical Systems Corp, Tokyo) and a high resolution endoscope video system (EVIS LUCERA ELITE CV-290/CLV-290SL, Olympus Medical Systems Corp, Tokyo) were used.

Note that the exclusion criteria of the endoscopic image are the same as that of the teacher data set, while all endoscopic images captured in the state where the esophagus is irradiated with white light or narrowband light were basically used for the purpose of avoiding bias. A Japan Gastroenterological Endoscopy Society preceptor prepared the evaluation test data set by specifically examining the prepared endoscopic images and determining the presence or absence of the multiple iodine unstained area.

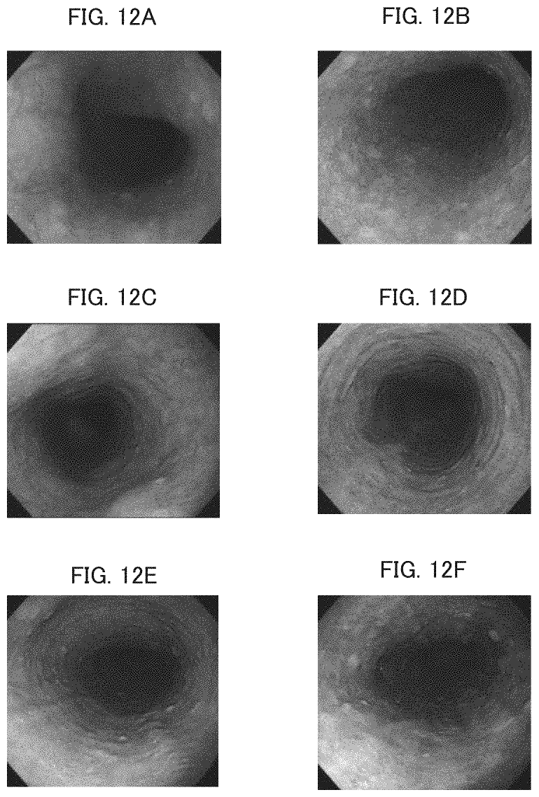

FIG. 12 is a diagram illustrating an example of an endoscopic image used for the evaluation test data set. FIG. 12A illustrates an endoscopic image captured with an endoscope capturing apparatus in the state where the esophagus of the subject is irradiated with white light, and determined that a multiple iodine unstained area is actually not present in the esophagus (the degree of staining when iodine staining is performed: grade A). FIG. 12B illustrates an endoscopic image captured with an endoscope capturing apparatus in the state where the esophagus of the subject is irradiated with narrowband light, and determined that a multiple iodine unstained area is actually not present in the esophagus (the degree of staining when iodine staining is performed: grade A).

FIG. 12C illustrates an endoscopic image captured with an endoscope capturing apparatus in the state where the esophagus of the subject is irradiated with white light, and determined that a multiple iodine unstained area is actually not present in the esophagus (the degree of staining when iodine staining is performed: grade B). FIG. 12D illustrates an endoscopic image captured with an endoscope capturing apparatus in the state where the esophagus of the subject is irradiated with narrowband light, and determined that a multiple iodine unstained area is actually not present in the esophagus (the degree of staining when iodine staining is performed: grade B).

FIG. 12E illustrates an endoscopic image captured with an endoscope capturing apparatus in the state where the esophagus of the subject is irradiated with white light, and determined that a multiple iodine unstained area is actually present in the esophagus (the degree of staining when iodine staining is performed: grade C). FIG. 12F illustrates an endoscopic image captured with an endoscope capturing apparatus in the state where the esophagus of the subject is irradiated with narrowband light, and determined that a multiple iodine unstained area is actually present in the esophagus (the degree of staining when iodine staining is performed: grade C).

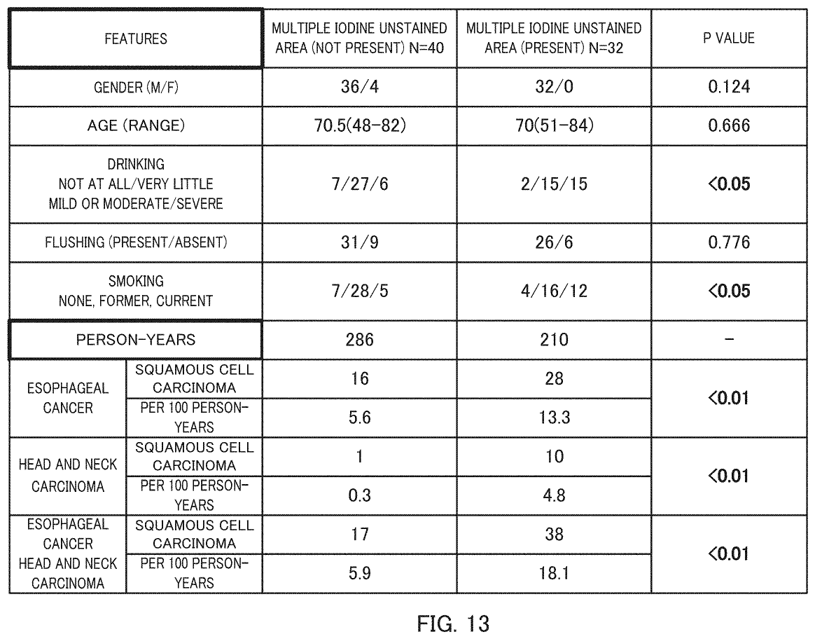

FIG. 13 is a diagram illustrating features of a subject related to the endoscopic image used for the evaluation test data set. For the age in FIG. 13, the center value is shown. For the comparison of various features between the subject actually not having a multiple iodine unstained area in the esophagus and the subject actually having a multiple iodine unstained area in the esophagus, Pearson's Chi-square test and Fisher's exact test were used, while Wald's test (see the P value of FIG. 13) was used for comparison of the person-years. Here, in each test, statistically significant difference was set to 0.05 or less. In this evaluation test, for the calculation of the P value, “EZR version 1.27 (Saitama Medical Center Jichi Medical University)” was used.

As illustrated in FIG. 13, the rate of heavy alcohol drinkers and currently smokers in the subjects with a multiple iodine unstained area in the esophagus is significantly higher than the subjects with no multiple iodine unstained area in the esophagus, while no significant difference in gender, age and flushing reaction was found therebetween. During the observation period, for the subject with no multiple iodine unstained area in the esophagus, the esophageal squamous cell carcinoma detected as simultaneous and heterochronic cancers per 100 person-years was 5.6, and the head and neck squamous cell carcinoma was 0.3. On the other hand, for the subject with a multiple iodine unstained area in the esophagus, the esophageal squamous cell carcinoma detected as simultaneous and heterochronic cancers per 100 person-years was 13.3, and the head and neck squamous cell carcinoma was 4.8.

### Method of Evaluation Test

In the present evaluation test, the evaluation test data set was input to the image diagnosis apparatus of the convolutional neural network base trained using the training data set, and whether a multiple iodine unstained area is present in the endoscopic image making up the evaluation test data set can be properly diagnosed (determined) was evaluated. The image diagnosis apparatus determines that a multiple iodine unstained area is present in the endoscopic image for the endoscopic image in which the degree of certainty of the presence or absence of the multiple iodine unstained area is greater than or equal to a predetermined value, whereas the image diagnosis apparatus determines that no multiple iodine unstained area is present in the endoscopic image for the endoscopic image in which the degree of certainty of the presence or absence of the multiple iodine unstained area is smaller than the predetermined value. The image diagnosis apparatus determines the presence of the multiple iodine unstained area for each endoscopic image, and determines the presence of the multiple iodine unstained area for each case on the basis of majority decision of the endoscopic image.

In addition, in the present evaluation test, to compare the diagnostic capability of the image diagnosis apparatus with the diagnostic capability of the endoscopist, the endoscopist made a diagnosis as to whether a multiple iodine unstained area is present in the endoscopic image by viewing the endoscopic image making up the evaluation test data set. As the endoscopist, ten endoscopists of Japan Gastroenterological Endoscopy Society with experience of 8 to 17 years as a doctor and endoscopic examinations of 3,500 to 18,000 cases were selected. The selected ten endoscopists made a diagnosis as to whether a multiple iodine unstained area is present for each endoscopic image, and made a diagnosis as to whether a multiple iodine unstained area is present for each case on the basis of majority decision of the endoscopic image.

In the present evaluation test, the sensitivity, specificity, positive predictive value (PPV), negative predictive value (NPV) and correct diagnosis rate with respect to the diagnostic capability of the image diagnosis apparatus (or endoscopist) were calculated by using the following expressions (5) to (9).

Sensitivity=(the number of cases where the presence of the multiple iodine unstained area in the esophagus has been properly diagnosed)/(the total number of cases where the multiple iodine unstained area is actually present in the esophagus) (5)

Specificity=(the number of cases where the non-presence of the multiple iodine unstained area in the esophagus has been properly diagnosed)/(the number of cases where the multiple iodine unstained area is actually not present in the esophagus total) (6)

Positive predictive value (PPV)=(the number of cases where a multiple iodine unstained area is actually present in the esophagus among cases diagnosed that a multiple iodine unstained area is present in the esophagus)/(the number of cases where diagnosed that multiple iodine unstained area is present in the esophagus) (7)

Negative predictive value (NPV)=(the number of cases where a multiple iodine unstained area is actually not present in the esophagus among cases diagnosed that the multiple iodine unstained area is not present in the esophagus)/(the number of cases diagnosed that the multiple iodine unstained area is not present in the esophagus) (8)

Correct diagnosis rate=(the number of cases where whether the multiple iodine unstained area is present in the esophagus has been properly diagnosed)/(the number of all cases) (9)

In addition, in the present evaluation test, the experienced endoscopist evaluated the presence/absence of the endoscopic findings of the background esophagus mucosa considered to be useful for properly diagnosing the presence of the multiple iodine unstained area for all endoscopic images making up the evaluation test data set, and made a diagnosis as to whether the multiple iodine unstained area is present in the esophagus for each endoscopic image on the basis of majority decision of the endoscopic findings. Then, regarding whether the presence of the multiple iodine unstained area in the esophagus can be properly diagnosed (sensitivity), the image diagnosis apparatus and the endoscopic findings are compared with each other to determine which is superior to the other.

The above-mentioned endoscopic findings are the following six findings, (a) to (f).

(a) Less than two glycogen acanthoses were identified in one visual field.

(b) Keratoderma (keratosis) is identified.

(c) Coarse (rough) esophagus mucosa is identified.

(d) No vascular translucency is identified when the esophagus is irradiated with white light.

(e) Erythrogenic background mucosa is identified when the esophagus is irradiated with white light.

(f) Brown background mucosa is identified when the esophagus is irradiated with narrowband light.

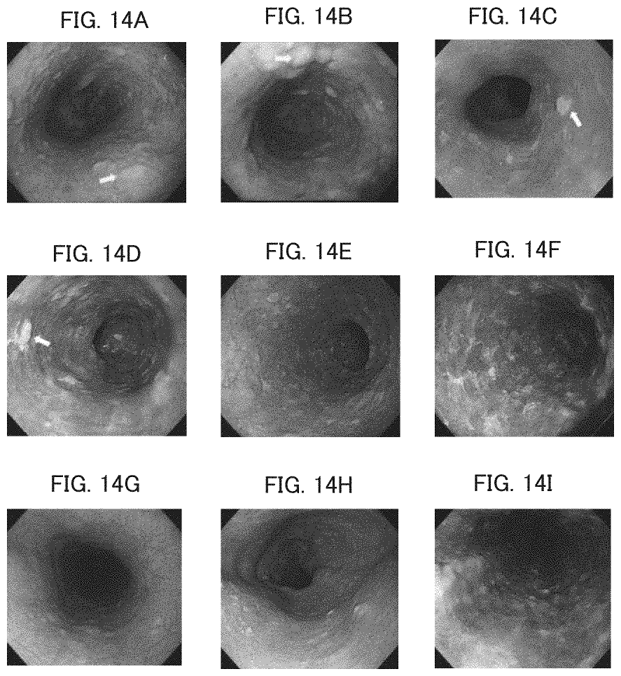

FIG. 14 is a diagram illustrating various endoscopic findings in an endoscopic image. FIG. 14A illustrates an endoscopic image where two ore more glycogen acanthoses are identified in one visual field, that is, the endoscopic finding (a) is not found when the esophagus is irradiated with white light. FIG. 14B illustrates an endoscopic image where two ore more glycogen acanthoses are identified in one visual field, that is, the endoscopic finding (a) is not found when the esophagus is irradiated with narrowband light. FIG. 14C illustrates an endoscopic image where keratoderma is identified, that is, the endoscopic finding (b) is found when the esophagus is irradiated with white light. FIG. 14D illustrates an endoscopic image where keratoderma is identified, that is, the endoscopic finding (b) is found when the esophagus is irradiated with narrowband light.

FIG. 14E illustrates an endoscopic image where a coarse esophageal mucosa is identified, that is, the endoscopic finding (c) is found when the esophagus is irradiated with white light. FIG. 14F illustrates an endoscopic image where a coarse esophageal mucosa is identified, that is, the endoscopic finding (c) is found when the esophagus is irradiated with narrowband light. FIG. 14G illustrates an endoscopic image where vascular translucency is identified, that is, the endoscopic finding (d) is not found when the esophagus is irradiated with white light. FIG. 14H illustrates an endoscopic image where an erythrogenic background mucosa is identified, that is, the endoscopic finding (e) is not found when the esophagus is irradiated with white light. FIG. 14I illustrates an endoscopic image where a brown background mucosa is identified, that is, the endoscopic finding (f) is found when the esophagus is irradiated with narrowband light.

### Result of Evaluation Test

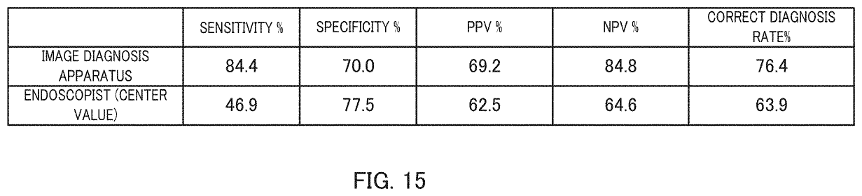

FIG. 15 is a diagram illustrating the sensitivity, specificity, positive predictive value, negative predictive value and correct diagnosis rate of an image diagnosis apparatus and an endoscopist. The sensitivity, specificity and correct diagnosis rate of the image diagnosis apparatus and the endoscopist were compared with each other by using two-sided McNemar test.

As illustrated in FIG. 15, the image diagnosis apparatus properly diagnosed the presence of the multiple iodine unstained area for 84.4% (=27/32) of the cases where the multiple iodine unstained area is present in the esophagus, and properly diagnosed the non-presence of the multiple iodine unstained area for 70.0% (=28/40) of the cases where the multiple iodine unstained area is not present in the esophagus. On the other hand, the endoscopist properly diagnosed the presence of the multiple iodine unstained area for 46.9% (=15/32) of the cases where the multiple iodine unstained area is present in the esophagus, and properly diagnosed the non-presence of the multiple iodine unstained area for 77.5% (=31/40) of the cases where the multiple iodine unstained area is not present in the esophagus. The correct diagnosis rate regarding the presence or absence of the multiple iodine unstained area was 76.4% in the image diagnosis apparatus and 63.9% in the endoscopist. In particular, the sensitivity of properly diagnosing the presence of the multiple iodine unstained area in the esophagus is significantly higher in the image diagnosis apparatus than than nine of ten endoscopists. On the other hand, there was no significant difference regarding the specificity and the correct diagnosis rate between the image diagnosis apparatus and endoscopist.

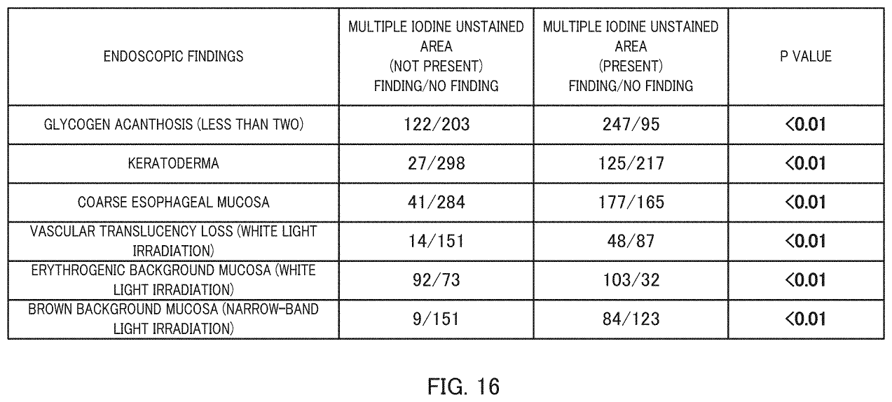

FIG. 16 is a diagram illustrating an evaluation result of the presence/absence of endoscopic findings for an endoscopic image with a multiple iodine unstained area and an evaluation result of the presence/absence of endoscopic findings for an endoscopic image with no multiple iodine unstained area, obtained by the endoscopist. Pearson's Chi-square test and Fisher's exact test were used for the comparison between the endoscopic image with a multiple iodine unstained area and the endoscopic image with no multiple iodine unstained area, regarding the number of positive evaluations for each of the endoscopic findings.

As illustrated in FIG. 16, in the endoscopic image where the multiple iodine unstained area is present in the esophagus, the number of positive evaluations of each of the findings of glycogen acanthosis (less than two), keratoderma, coarse esophageal mucosa, loss of vascular translucency, erythrogenic background mucosa and brown background mucosa is significantly greater than that of the endoscopic image with no multiple iodine unstained area. That is, when it is evaluated that there is an endoscopic finding, the possibility of the presence of the multiple iodine unstained area in the esophagus can be considered to be high to a certain degree.

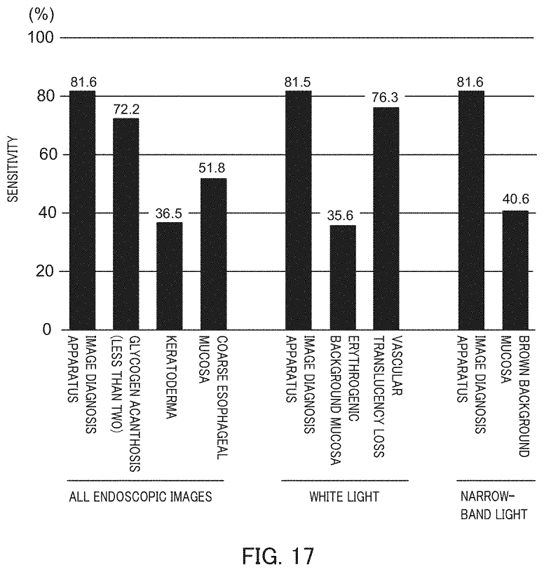

FIG. 17 is a diagram illustrating a result of comparison between the image diagnosis apparatus and the endoscopic findings regarding whether the presence of the multiple iodine unstained area in the esophagus can be properly diagnosed (sensitivity) with reference to an endoscopic image. A two-sided McNemar test was used for the comparison of the sensitivity between the image diagnosis apparatus and each endoscopic finding.

As illustrated in FIG. 17, in all endoscopic images (white light observation and narrowband light observation), the sensitivity of the image diagnosis apparatus was 81.6% (=279/342), and the presence of the multiple iodine unstained area was properly diagnosed far more than the case evaluated that there is an endoscopic finding regarding glycogen acanthosis (less than two), keratoderma, and coarse esophageal mucosa. In the endoscopic image obtained by irradiating the esophagus with white light, the sensitivity of the image diagnosis apparatus was 81.5% (=110/135), and the presence of the multiple iodine unstained area was properly diagnosed far more than the case evaluated that there is an endoscopic finding regarding the erythrogenic background mucosa. In the endoscopic image obtained by irradiating the esophagus with narrowband light, the sensitivity of the image diagnosis apparatus was 81.6% (=169/207), and the presence of the multiple iodine unstained area was properly diagnosed far more than the case evaluated that there is an endoscopic finding regarding brown background mucosa. As described above, the image diagnosis apparatus achieves higher sensitivity than the cases positively evaluated regarding the endoscopic findings, and the sensitivity was highest in the case positively evaluated regarding the finding of “loss of vascular translucency” among the endoscopic findings.

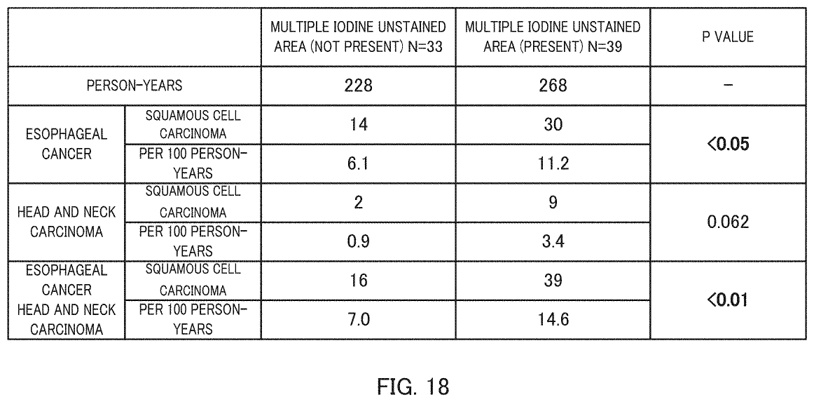

FIG. 18 is a diagram illustrating the numbers of esophageal squamous cell carcinomas and head and neck squamous cell carcinomas detected as simultaneous and heterochronic cancers for a case diagnosed with an image diagnosis apparatus that a multiple iodine unstained area is present (not present) in the esophagus. A case diagnosed that a multiple iodine unstained area is present and a case diagnosed that no multiple iodine unstained area is present were compared with each other by using Pearson's Chi-square test and Fisher's exact test.

As illustrated in FIG. 18, in a case diagnosed that a multiple iodine unstained area is present in the esophagus with an image diagnosis apparatus, the number of esophageal squamous cell carcinomas detected per 100 person-years was 11.2, and the number of esophageal squamous cell carcinomas and head and neck squamous cell carcinomas was 14.6. In a case diagnosed that that there is no multiple iodine unstained area in the esophagus is present with the image diagnosis apparatus, the number of esophageal squamous cell carcinomas detected per 100 person-years was 6.1, and the number of esophageal squamous cell carcinomas and head and neck squamous cell carcinomas was 7.0. In this manner, for the esophageal squamous cell carcinoma, and for the esophageal squamous cell carcinoma and the head and neck squamous cell carcinoma, the incidence rate as simultaneous and heterochronic cancers was significantly higher in the case diagnosed that a multiple iodine unstained area is present in the esophagus than the case diagnosed that no multiple iodine unstained area is present in the esophagus. Thus, the image diagnosis apparatus achieves stratification of the risk of the esophageal squamous cell carcinoma and the head and neck squamous cell carcinoma as the simultaneous and heterochronic cancers, in addition to the presence or absence of the multiple iodine unstained area in the esophagus.

### Considerations for Second Evaluation Test

As described above, the image diagnosis apparatus achieved a diagnosis of the presence or absence of the multiple iodine unstained area, which is an indicator of the high-risk case of the esophageal squamous cell carcinoma and the head and neck squamous cell carcinoma, in the endoscopic image obtained by capturing the esophagus where no iodine staining has been performed, with a sensitivity higher than that of experienced endoscopists by using the diagnostic capability of the endoscopic image of the convolutional neural network.

Today, known risk factors of esophageal squamous cell carcinoma include heavy alcohol consumption, smoking, flushing reaction, and the like. The endoscopic findings of the multiple iodine unstained area recognized after performing iodine staining on the esophagus reflect all of the above-mentioned risk factors, stratifying the risk of the esophageal squamous cell carcinoma and the head and neck squamous cell carcinoma. The multiple iodine unstained area is also very useful for determining the schedule of the surveillance (periodical inspection) after the treatment of the esophageal squamous cell carcinoma and the head and neck squamous cell carcinoma. However, because the presence or absence of the multiple iodine unstained area cannot be determined when the iodine staining is not performed, the iodine staining is normally used only for cancers or suspected cancer lesions, and as such the usability is limited. However, by using the image diagnosis apparatus, the risk of the esophageal squamous cell carcinoma can be determined from endoscopic images captured without performing iodine staining in the first endoscope inspection (EGD) in all subjects.

For high-risk cases with high risk of esophageal squamous cell carcinoma and head and neck squamous cell carcinoma, it is ideal to carefully observe the esophagus and pharynx under irradiation with narrowband light and observe the esophagus with the iodine staining being performed, but it is not realistic to perform the iodine staining in all cases. Iodine staining is used for those with cancer or suspected cancer for the purpose of picking up cancer without missing it and diagnosing the extent of the cancer. In addition, the cancer risk can be determined based on the degree of multiple iodine unstained area. It should be noted that it cannot be used for patients with iodine allergy because it is irritating and causes discomfort. It would be more useful if AI could be used without performing iodine staining to determine the cancer risk and recognize high-risk cases from endoscopic images of the esophagus. However, there is no known endoscope inspection method for effectively determining the multiple iodine unstained area from the esophageal endoscopic image with no iodine staining performed, and the present invention has accomplished it the first time.

In view of this, in the present evaluation test, to diagnose the presence or absence of the multiple iodine unstained area from the esophageal endoscopic image with no iodine staining performed, the presence/absence of six endoscopic findings were evaluated. Each of these endoscopic findings is frequently confirmed in a case where multiple iodine unstained area is present. In particular, the sensitivity of the two endoscopic findings “less than two glycogen acanthoses were identified” and “no vascular translucency was identified when the esophagus is irradiated with white light” is higher than expected, and thus the presence or absence of the multiple iodine unstained area can be diagnosed from the esophageal endoscopic image with no iodine staining performed. However, regarding properly diagnosing the presence of the multiple iodine unstained area, the sensitivity of the endoscopist was as low as 46.9% (see FIG. 15). One possible reason for this is that many endoscopists did not confirm the above-mentioned two endoscopic findings. Then, the sensitivities of other four endoscopic findings were low. On the other hand, the sensitivity of the image diagnosis apparatus was higher than that of the six endoscopic findings, and than that of the experienced endoscopists. This suggests that the image diagnosis apparatus is superior to human endoscopists in diagnosing the presence or absence of the multiple iodine unstained area by comprehensively determining the endoscopic findings.

In addition, the evaluation test data set was used to examine the diagnostic capability of “multiple foci of dilated vessels (MDV)” reported by Matsuno et al. The inventor had limited knowledge of MDVs, and therefore recognizing MDVs from unmagnified still images was a bit difficult. Although a little more training is considered necessary to compare the results with other knowledge, the present inventor's analysis showed that MDV had a sensitivity of 59.4%, a specificity of 70.4%, and an accuracy of 79.5%. In other words, MDV showed high specificity and accuracy in the original paper, but sensitivity was not as high in the original paper as in the inventor's analysis. To recognize more high-risk cases of esophageal squamous cell carcinoma and head and neck squamous cell carcinoma and to ensure that esophageal squamous cell carcinoma and head and neck squamous cell carcinoma are not missed, the sensitivity value having the highest value in the imaging system is considered the most important diagnostic value.

As described above, the present inventor constructed the image diagnosis apparatus capable of diagnosing cases where multiple iodine unstained area is present, that is, cases with high risks of esophageal squamous cell carcinoma and head and neck squamous cell carcinoma, with high sensitivity from the esophageal endoscopic image with no iodine staining performed. With this image diagnosis apparatus, the endoscopist can efficiently detect high-risk cases of esophageal squamous cell carcinoma requiring careful surveillance in typical endoscope inspection using no iodine staining, and can perform highly accurate esophageal cancer diagnosis by appropriately applying iodine staining.

This application is entitled to and claims the benefit of Japanese Patent Application No. 2020-078601 filed on Apr. 27, 2020, the disclosure each of which including the specification, drawings and abstract is incorporated herein by reference in its entirety.

### INDUSTRIAL APPLICABILITY

The present invention is useful as an image diagnosis apparatus, an image diagnosis method, an image diagnosis program and a learned model that can improve the diagnosis accuracy of esophageal cancer in esophageal endoscope inspection. The cancer risk is also determined through real time video diagnosis and estimation of multiple iodine unstained area, and thus a quick and highly accurate endoscopic esophageal cancer diagnosis method suitable for each the subject's organ is provided.

### REFERENCE SIGNS LIST

10, 10A Endoscopic image acquisition section
20, 20A Estimation section
30, 30A Display control section
40, 40A Learning apparatus
100, 100A Image diagnosis apparatus
101 CPU
102 ROM
103 RAM
104 External storage apparatus
105 Communication interface
200, 200A Endoscope capturing apparatus
300, 300A Display apparatus
D1 Endoscopic image data
D2 Estimation result data
D3 Determination result image data
D4 Training data

1. An image diagnosis apparatus, comprising:
an endoscopic image acquisition section configured to acquire an endoscope video obtained by capturing an esophagus of a subject;
an estimation section configured to estimate a position of an esophageal cancer present in the endoscope video acquired by using a convolutional neural network trained as training data with an esophageal cancer image obtained by capturing an esophagus where an esophageal cancer is present; and
a display control section configured to display the position of the esophageal cancer estimated and a degree of certainty indicating a possibility of presence of the esophageal cancer at the position on the endoscope video in a superimposed manner.

2. The image diagnosis apparatus according to claim 1,
wherein the endoscope video is captured by inserting an endoscope capturing apparatus to the esophagus; and
wherein the image diagnosis apparatus further comprises an alert output control section configured to set a reference insertion speed of the endoscope capturing apparatus as an observation speed of a lumen of the esophagus corresponding to a risk of presence of an esophageal cancer in the esophagus, and output an alert upon a discrepancy between the reference insertion speed and an actual insertion speed.

3. The image diagnosis apparatus according to claim 2, wherein the risk is determined based on estimation of presence or absence of a multiple iodine unstained area in the esophagus by using a convolutional neural network trained with a multiple iodine unstained area esophagus image and a non-multiple iodine unstained area esophagus image as training data, the multiple iodine unstained area esophagus image being a non-iodine staining image obtained by capturing an esophagus where a multiple iodine unstained area is present without performing iodine staining, the non-multiple iodine unstained area esophagus image being a non-iodine staining image obtained by capturing an esophagus where no multiple iodine unstained area is present without performing iodine staining.

4. An image diagnosis method, comprising:
acquiring an endoscope video obtained by capturing an esophagus of a subject;
estimating a position of an esophageal cancer present in the endoscope video acquired by using a convolutional neural network trained as training data with an esophageal cancer image obtained by capturing an esophagus where an esophageal cancer is present; and
displaying the position of the esophageal cancer estimated and a degree of certainty indicating a possibility of presence of the esophageal cancer at the position on the endoscope video in a superimposed manner.

5. The image diagnosis method according to claim 4, wherein the convolutional neural network trained with the esophageal cancer image as training data is executed with the convolutional neural network coupled with a convolutional neural network trained as training data with a multiple iodine unstained area esophagus image and a non-multiple iodine unstained area esophagus image, the multiple iodine unstained area esophagus image being a non-iodine staining image obtained by capturing an esophagus where a multiple iodine unstained area is present without performing iodine staining, the non-multiple iodine unstained area esophagus image being a non-iodine staining image obtained by capturing an esophagus where no multiple iodine unstained area is present without performing iodine staining.

6. An image diagnosis program configured to cause a computer to execute:
an endoscopic image acquisition process of acquiring an endoscope video obtained by capturing an esophagus of a subject;
an estimation process of estimating a position of an esophageal cancer present in the endoscope video acquired by using a convolutional neural network trained as training data with an esophageal cancer image obtained by capturing an esophagus where an esophageal cancer is present; and
a display control process of displaying the position of the esophageal cancer estimated and a degree of certainty indicating a possibility of presence of the esophageal cancer at the position on the endoscope video in a superimposed manner.

7. The image diagnosis method according to claim 6, wherein the convolutional neural network trained with the esophageal cancer image as training data is executed with the convolutional neural network coupled with a convolutional neural network trained as training data with a multiple iodine unstained area esophagus image and a non-multiple iodine unstained area esophagus image, the multiple iodine unstained area esophagus image being a non-iodine staining image obtained by capturing an esophagus where a multiple iodine unstained area is present without performing iodine staining, the non-multiple iodine unstained area esophagus image being a non-iodine staining image obtained by capturing an esophagus where no multiple iodine unstained area is present without performing iodine staining.

8. A learned model obtained through learning of a convolutional neural network with a multiple iodine unstained area esophagus image and a non-multiple iodine unstained area esophagus image as teacher data, the multiple iodine unstained area esophagus image being a non-iodine staining image obtained by capturing an esophagus where a multiple iodine unstained area is present without performing iodine staining, the non-multiple iodine unstained area esophagus image being a non-iodine staining image obtained by capturing an esophagus where no multiple iodine unstained area is present without performing iodine staining,
the learned model being configured to cause a computer to estimate whether there is an association between an endoscopic image obtained by capturing an esophagus of a subject and an esophageal cancer, and output an estimation result.
